(19) United States
(12) Patent Application Publication
(10) Pub. No.: US 2016/0318961 A1
(43) Pub. Date: Nov. 3, 2016

(54) COMPLEXES AND CATALYTIC PROCESSES

(71) Applicant: THE UNIVERSITY OF SUSSEX, Brighton, Sussex (GB)

(72) Inventor: Oscar Navarro Fernandez, Brighton, Sussex (GB)

(21) Appl. No.: 15/103,181

(22) PCT Filed: Dec. 9, 2014
(86) PCT No.: PCT/GB2014/053639

§ 371 Date: Jun. 9, 2016

(30) Foreign Application Priority Data
Dec. 9, 2013 (GB) 1321706.2

(51) Int. Cl. C07F 15/00; C07C 45/29; C07C 17/278; C07D 213/26; B01J 31/22; C07C 45/64; C07D 213/30; C07D 213/74; C07C 41/30; C07C 211/63; C07C 45/62

(52) U.S. Cl. CPC C07F 15/006; C07C 211/63; C07C 45/29; C07C 17/278; C07D 213/26; C07C 45/62; C07C 45/64; C07D 213/30; C07D 213/74; C07C 41/30; B01J 31/2295; B01J 2231/763; B01J 2231/44; B01J 2231/40; B01J 2231/645; B01J 2231/49; B01J 2531/824

(57) ABSTRACT

The present application is directed towards complexes of formula (I), to methods for preparing such complexes, and to use of such complexes in catalysis. The complexes show utility in a range of catalytic cycles, including Pd(ll)/Pd(IV) cycles. (Formula (I))

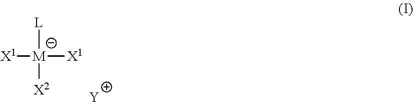

The present invention relates to complexes of formula (I), and to methods for the preparation of said complexes. In particular, the complexes of the present invention are useful catalysts in carbon-carbon and carbon-heteroatom bond-forming reactions, oxidation reactions, tandem organic/organometallic catalysis and enantioselective catalysis.

### BACKGROUND OF THE INVENTION

Metal, and particularly palladium, complexes have received much attention in recent years as potential catalysts for carbon-carbon and carbon-heteroatom bond-forming reactions.

Many of the metal complexes known in the art for performing such reactions have a number of disadvantages. In particular, they can be unstable when handled in air and/or moisture, which significantly restricts their use on a commercial scale. In addition, unwanted side reactions may occur that could deactivate the catalyst. This effectively leads to “loss” of the metal, which is often a precious or semi-precious metal such as platinum or palladium. Consequently there is a need in the art for new metal complexes that have improved air and/or moisture stability.

Many of the complexes known in the art catalyse C—H bond activation and subsequent carbon-carbon and carbon-heteroatom bond forming reactions using transitions between the M⁰ and M^{II} oxidation states, where M is the metal ion at the core of the complex. However, metal complexes that use the M^{II}/M^{IV} oxidation states in such catalytic processes are also known.

Protocols known in the art for catalysing C—H bond activation reactions using the M^{II}/M^{IV} catalytic cycle tend to use Pd(OAc)₂ catalysts. However, these reactions are frequently performed at high temperatures, which can lead to degradation of the catalyst and unwanted side reactions (see pages 5099-5100 of Chen et al., Angew. Chem. Int. Ed., 2009, 48, 5094-5115).

Tandem catalysis describes coupled catalyses in which sequential transformation of the substrate occurs via two (or more) mechanically distinct processes, with all catalytic species—whether masked or apparent—present from the outset. Tandem catalysis is also known as domino or cascade catalysis. One key advantage of tandem catalysis is the elimination of intermediate work-up and isolation steps, which can both speed up synthesis and improve yield. However, catalyst recovery can be problematic and efficiency may be low (Fogg & Santos, Coord. Chem. Rev., 2004, 248, 2365). Improved complexes for tandem catalysis are therefore sought.

Metal complexes may also be used in enantioselective catalysis. Control of stereochemistry in chemical reactions is of critical importance in many fields of chemistry, and particularly in the area of pharmaceutical research. Many reactions produce mixtures of enantiomers, which must be separated. Resolution of enantiomers is a difficult task (and is sometimes impossible). Consequently, there has been much effort into developing methods for controlling the stereochemical outcome of a reaction.

One approach is the use of so called “chiral auxiliaries”. Chiral auxiliaries are chiral chemical compounds that are temporarily incorporated into a reaction in order to control the stereochemical outcome of that reaction. Thus, the chiral auxiliary is incorporated into the substrate and must subsequently be cleaved from the final product. The chiral auxiliary can then be recovered for future use. While this is a useful synthetic transformation, the obvious drawback is the need to cleave the chiral auxiliary from the substrate. This introduces additional steps into the synthesis, leading to higher costs and longer synthesis times.

Alternative approaches to control the stereochemical outcome of a reaction include biocatalysis and chiral pool synthesis. Biocatalysis makes use of biological compounds, such as enzymes, to perform chemical transformations. Often biocatalysis results in very high enantioselectivity. However, the specificity of biocatalysts makes them unsuitable for a wide variety of substrates. Chiral pool synthesis involves manipulation of chiral starting material by altering the stereochemistry of a starting material using a non-chiral molecule e.g. inverting the stereochemistry of a carbon atom via an S_{N}2 reaction. Chiral pool synthesis is attractive for target molecules having similar chirality to a naturally occurring substance. However, a stoichiometric amount of the enantiopure starting material is required, which can be expensive.

It has been found that chiral metal complexes can be used to induce stereoselectivity in organic reactions. For example, Kang et al. demonstrated catalytic enantioselective Diels-Alder reactions using chiral palladium complexes (Bull. Korean Chem. Soc., 2008, Vol. 29, no. 11, 2093-2094). However, new metal complexes are sought that reduce problems associated with air/moisture stability of such compounds, as well as complexes that have better enantioselectivity.

### SUMMARY OF THE INVENTION

In one aspect, the present invention provides a complex of formula (I):

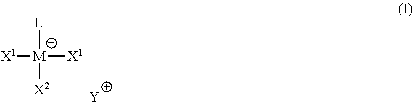

wherein:

M denotes a metal selected from Pd, Pt and Ni;

L denotes an N-heterocyclic carbene ligand;

X¹ denotes F, Cl, Br, I, OH or alkoxide;

X² denotes F, Cl, Br, I, CN, SCN, NCS, N₃ or R^{x}CO₂;

R^{x} denotes H or C₁-C₄ alkyl; and

Y denotes a non-coordinating cation.

In another aspect, the present invention provides a method for the preparation of a complex of formula (I) comprising reacting a complex of formula (II) with X²Y in a polar solvent:

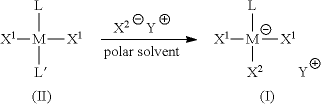

wherein:

M denotes a metal selected from Pd, Pt and Ni;

L denotes an N-heterocyclic carbene ligand;

X¹ denotes F, Cl, Br, I, OH or alkoxide;

X² denotes F, Cl, Br, I, CN, SCN, NCS, N₃ or R^{x}CO₂;

R^{x} denotes H or C₁-C₄ alkyl;

Y denotes a non-coordinating cation; and

L′ denotes a tertiary amine.

In a further aspect, the present invention relates to reactions using a complex of formula (I). In particular, complexes of formula (I) are active as catalysts in a number of reactions. Thus, the present invention further relates to the use of a complex of formula (I) as a catalyst, and to catalytic reactions using a complex of formula (I).

### DESCRIPTION OF THE INVENTION

The complexes of formula (I) contain an N-heterocyclic carbene ligand.

By “N-heterocyclic carbene ligand” is meant a heterocyclic ring containing five atoms with nitrogen atoms adjacent to a carbon which is neutral and has a valence of 2.

Preferably, the N-heterocyclic carbene ligand is an imidazol-2-ylidene, a tetrahydroimidazol-2-ylidene, or a triazol-5-ylidene.

Thus, preferably the present invention relates to a complex of formula (Ia)

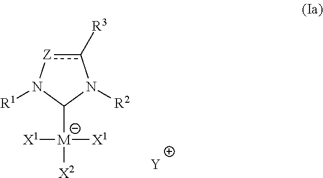

wherein
M denotes a metal selected from Pd, Pt and Ni;
X¹ denotes F, Cl, Br, I, OH or alkoxide;
X² denotes F, Cl, Br, I, CN, SCN, NCS, N₃ or R^{x}CO₂;
R^{x} denotes H or C₁-C₄ alkyl;
R¹ and R² independently denote C₁-C₄-alkyl, C₃-C₁₀ cyclohexyl; or C₆-C₁₀ aryl optionally substituted with 1 to 5 R^{a};
Z denotes N, CR⁴ or CHR⁴;
R³ and R⁴ independently denote H, alkyl, aryl, cycloalkyl, alkylaryl, alkyl cycloalkyl, which may be optionally substituted by one or more R^{a}; or
R³ and R⁴ together form a 5-7 membered ring, which may be saturated or unsaturated; or
R² and R³ and/or R¹ and R⁴ may together form an alkylene group having 3 or 4 carbon atoms;
each R^{a} independently denotes halogen, NO₂, C₁-C₄-alkyl, or C₁-C₄-alkoxy;
   and
Y denotes a non-coordinating cation.

In the complex of formula (Ia), the Z moiety denotes whether the dashed line represents a single or a double bond. Thus, when Z denotes N or CR⁴, the dashed line is a double bond, and when Z denotes CHR⁴, the dashed line represents a single bond, as shown below:

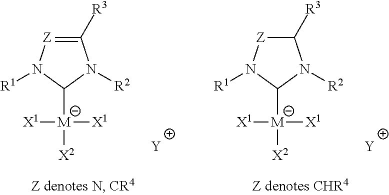

In an analogous manner, complexes of formula (Ia) may be formed by reacting an analogous complex of formula (IIa) with an X²Y salt in a polar solvent, as shown below:

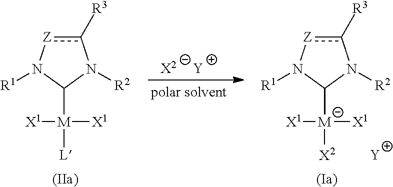

wherein M, Z, R¹, R², R³, X¹, X², L′ and Y are as defined above.

The complexes of formula (II) and (IIa) may be formed by reaction of a metal dimer with L′, as shown below:

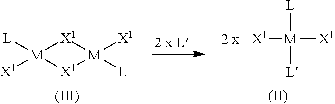

These types of reactions are known in the art, for example from Chem. Eur. J., 2006, 12, 4749-4755; J. Am. Chem. Soc., 2006, 128, 4101-4111; Organometallics, 2004, 23, 1629-1635; J. Am. Chem. Soc., 2007, 129, 7274-7276; J. Am. Chem. Soc., 2008, 130, 13552-13554; Chem. Eur. J., 2009, 15, 4281-4288; Eur. J. Org. Chem., 2010, 4345-4354; Synthesis, 2008, 2776-2797; Chem. Eur. J., 2010, 16, 10844-10853; Chem. Commun., 2008, 735-737; and Angew. Chem. Int. Ed., 2011, 50, 3896-3899.

Complexes of formula (III) are known in the art, and would be easily obtainable for the skilled person.

The complex of formula (II) and the resultant complex of formula (I) are catalytically active, which gives rise to the possibility of side reactions taking place. However, advantageously the method can be carried out at moderate temperatures, thus reducing the likelihood of side reactions (such as activation of carbon-hydrogen bonds in the solvent) taking place.

Accordingly, the method of the invention is preferably performed at temperatures of 40° C. or below, more preferably 35° C. or below, even more preferably 30° C. or below.

In other words, the temperature throughout the method including the step of reacting the complex of formula (II) with X²Y in a polar solvent is controlled so as to be 40° C. or below, or more preferably any of the other preferred temperatures mentioned above.

Preferably, the method of the invention is performed at “room temperature”, i.e. between 15 and 25° C.

Thus, preferred temperature ranges for carrying out the method of the invention include from −20° C. to 40° C.; from −10° C. to 40° C.; from 0° C. to 35° C.; from 10° C. to 30° C.; and from 15° C. to 25° C.

The at least one solvent used in the method of the invention is selected so that both X²⁻Y⁺ and the metal complex are in solution at room temperature, i.e. between 15 and 25° C. It is often necessary for mixtures of solvents to be used to ensure that the components are fully solubilised. While it is not necessary for the solvents to be anhydrous, this is preferred.

Preferred solvents are selected from alcohols, ethers, ketones, aldehydes, esters and amides.

Preferred alcohol solvents include methanol, ethanol, n-propanol, iso-propanol, n-butanol, sec-butanol, iso-butanol and tert-butanol. Methanol is particularly preferred.

Preferred ether solvents include 1,4-dioxane, tetrahydrofuran, di-tert-butyl ether, diethyl ether, diethylene glycol diethyl ether, diglyme, diisopropyl ether, dimethoxyethane, di methoxymethane, ethyl tert-butyl ether, methoxyethane, 2-(2-methoxyethoxy)ethanol, methyl tert-butyl ether, 2-methyltetrahydrofuran, morpholine, and tetrahydropyran. 1,4-Dioxane and tetrahydrofuran are particularly preferred.

Preferred ketone solvents include acetone, acetophenone, butanone, ethyl isopropyl ketone, isophorone, mesityl oxide, methyl isobutyl ketone, methyl isopropyl ketone, and 3-pentanone. Acetone is a particularly preferred ketone solvent.

Preferred aldehyde solvents include acetaldehyde, propionaldehyde, n-butyraldehyde and isobutyraldehyde.

Preferred ester solvents include benzyl benzoate, bis(2-ethylhexyl) adipate, bis(2-ethylhexyl) phthalate, butyl acetate, sec-butyl acetate, tert-butyl acetate, diethyl carbonate, dioctyl terephthene, ethyl acetate, ethyl acetoacetate, ethyl butyrate, ethyl lactate, ethylene carbonate, hexyl acetate, isoamyl acetate, isobutyl acetate, isopropyl acetate, methyl acetate, methyl phenylacetate, methyl propionate, propyl acetate, propylene carbonate and triacetin.

Preferred amide solvents include formamide, N,N-dimethyl formamide, N-methyl formamide, dimethyl acetamide and 2-pyrrolidone.

Thus, a particularly preferred set of solvents is selected from methanol, 1,4-dioxane, tetrahydrofuran, acetone, and mixtures thereof, with 1,4-dioxane, tetrahydrofuran, acetone, and mixtures thereof being the most preferred.

The method of the invention is preferably carried out at atmospheric pressure (i.e. about 101325 Pa). Use of elevated pressures may result in unwanted side reactions.

The time taken to form the complex of formula using the method of the invention will vary depending on a number of factors, which include, but are not limited to, the reactivity of the starting complex, the solubility of the starting complex and salt X²⁻Y⁺, the solvent(s) that are used and the temperature at which the method is carried out. Typically, to ensure that the reaction is complete, the method of the invention is performed over 12-24 hours, with stirring, to ensure even mixing of reactants.

However, the method of the invention may take from 1 minute to 24 hours; for example from 5 minutes to 16 hours; from 15 minutes to 8 hours; from 30 minutes to 4 hours; from 1 to 2 hours.

In some embodiments, longer reaction times may be needed. Thus, the method of the invention may take from 2 hours to 24 hours, preferably from 4 hours to 24 hours, more preferably from 8 hours to 24 hours, more preferably from 12 hours to 24 hours.

Depending on the relative solubility of the complex of formula, it may precipitate out of the reaction mixture, and therefore be recovered by simple filtration. If precipitation does not occur, and L′ is reasonably volatile (i.e. has a boiling point at atmospheric pressure of about 150° C. or below), the complex of formula may be recovered by simultaneous evaporation of both the solvent and amine. If precipitation does not occur, and L′ is not volatile, the complex of formula may be recovered from the reaction mixture by standard purification techniques known in the art, such as chromatography.

The method of the invention involves the displacement of the L′ ligand by the anion X². As noted above, L′ is a tertiary amine.

Preferably,
L′ denotes NR′R″R′″;
wherein
R′, R″ and R′″ each independently denote alkyl, cycloalkyl or aryl, which may be optionally substituted by one or more R^{a}; or
together two of R′ and R″ combine to form a ring containing 4-6 alkylene units; or
R′, R″ and R′″ together with the N combine to form quinuclidine (1-azabicyclo[2.2.2]octane) or DABCO (1,4-diazabicyclo[2.2.2]octane); and
R^{a} is as defined above for the complex of formula (Ia).

Particularly preferred definitions of the various substituents are provided below, any two or more of which can be combined to provide further preferred embodiments of the invention. In particular, any of the preferred definitions of the cation Y may be combined with any of the preferred definitions of the anionic metal complexes as shown below.

Thus, preferably in the complexes of formula (Ia) and (IIa),
R³ and R⁴ independently denote H, C₁-C₄ alkyl, C₆-C₁₀ aryl, C₃-C₆ cycloalkyl, C₁-C₄-alkyl-C₆-C₁₀-aryl, C₁-C₄-alkyl-C₃-C₆-cycloalkyl, which may be optionally substituted by one or more R^{a}; or
R³ and R⁴ together form a 5-7 membered ring, which may be saturated or unsaturated; or
R² and R³ and/or R¹ and R⁴ may together form an alkylene group having 3 or 4 carbon atoms.

Preferably, in the complexes of formula (Ia) and (IIa),
R³ and R⁴ independently denote H or C₁-C₄ alkyl; or
R³ and R⁴ together form a 5-7 membered ring, which may be saturated or unsaturated; or
R² and R³ and/or R¹ and R⁴ may together form an alkylene group having 3 or 4 carbon atoms.

Preferably, in the complexes of formula (Ia) and (IIa),
Z denotes CHR⁴ or CR⁴.

Preferably, in the complexes of formula (Ia) and (IIa),
Z denotes CR⁴.

Preferably, in the complexes of formula (Ia) and (IIa),
Z denotes CHR⁴ or CR⁴;
R³ and R⁴ independently denote H, C₁-C₄ alkyl, C₆-C₁₀ aryl, C₃-C₆ cycloalkyl,
C₁-C₄-alkyl-C₃-C₆-cycloalkyl, which may be optionally substituted by one or more R^{a}; or
R³ and R⁴ may together form —(CH)₄— when Z denotes CR⁴.

Preferably, in the complexes of formula (Ia) and (IIa),
Z denotes CR⁴;
R³ and R⁴ independently denote H, C₁-C₄ alkyl, C₁-C₄-alkyl-C₆-C₁₀-aryl, C₁-C₄-alkyl-C₃-C₆-cycloalkyl, which may be optionally substituted by one or more R^{a}; or
R³ and R⁴ may together form —(CH)₄— when Z denotes CR⁴.

Preferably, in the complexes of formula (Ia) and (IIa),
Z denotes CR⁴;
R³ and R⁴ independently denote H or C₁-C₄ alkyl; or
R³ and R⁴ may together form —(CH)₄— when Z denotes CR⁴.

Preferably, in the complexes of formula (Ia) and (IIa),
R¹ and R² independently denote C₁-C₄-alkyl, C₃-C₁₀ cycloalkyl; or phenyl optionally substituted with 1 to 5 R^{a}.

Preferably, in the complexes of formula (Ia) and (IIa),
R¹ and R² independently denote C₁-C₄-alkyl, cyclohexyl, adamantyl; or phenyl optionally substituted with 1 to 5 R^{a}.

Preferably, in the complexes of formula (Ia) and (IIa),
R¹ and R² independently denote isopropyl, tert-butyl, cyclohexyl, adamantyl; or phenyl optionally substituted with 1 to 5 R^{a}.

Preferably, in the complexes of formula (Ia) and (IIa),
R¹ and R² independently denote isopropyl, tert-butyl, cyclohexyl, adamantyl; or phenyl optionally substituted with 1-3 methyl groups.
R¹ and R² independently denote isopropyl, tert-butyl, cyclohexyl, adamantyl; or phenyl optionally substituted with methyl groups in the 2, 4 and 6 positions.

Preferably, in the complexes of formula (I), (Ia), (II) and (IIa),
M denotes Pd or Pt.

Preferably, in the complexes of formula (I), (Ia), (II) and (IIa),
M denotes Pd.

Preferably, in the complexes of formula (I), (Ia), (II) and (IIa),
X¹ denotes F, Cl, Br, I, OH, or C₁-C₄-alkoxide.

Preferably, in the complexes of formula (I), (Ia), (II) and (IIa),
X¹ denotes Cl, Br, I, OH, or alkoxide.

Preferably, in the complexes of formula (I), (Ia), (II) and (IIa),
X¹ denotes Cl, Br, I, OH, or C₁-C₄-alkoxide.

Preferably, in the complexes of formula (I), (Ia), (II) and (IIa),
X¹ denotes F, Cl, Br, I, or C₁-C₄-alkoxide.

Preferably, in the complexes of formula (I), (Ia), (II) and (IIa),
X¹ denotes Cl, Br, I, or C₁-C₄-alkoxide.

Preferably, in the complexes of formula (I), (Ia), (II) and (IIa),
X¹ denotes F, Cl or Br.

Preferably, in the complexes of formula (I), (Ia), (II) and (IIa),
X¹ denotes Cl or Br.

Preferably, in the complexes of formula (I), (Ia), (II) and (IIa),
X¹ denotes Cl.

Preferably, in the complexes of formula (I) or (Ia) (and in the method of the invention),
X² denotes F, Cl, Br, I, CN, SCN, NCS, N₃ or R^{x}CO₂.

Preferably, in the complexes of formula (I) or (Ia) (and in the method of the invention),
X² denotes Cl, Br, I, CN, SCN, NCS, N₃ or R^{x}CO₂.

Preferably, in the complexes of formula (I) or (Ia) (and in the method of the invention),
X² denotes F, Cl, Br, I, or CN.

Preferably, in the complexes of formula (I) or (Ia) (and in the method of the invention),
X² denotes Cl, Br, I, or CN.

Preferably, in the complexes of formula (I) or (Ia) (and in the method of the invention),
X² denotes F, Cl, Br, or I.

Preferably, in the complexes of formula (I) or (Ia) (and in the method of the invention),
X² denotes Cl, Br, or I.

Preferably, in the complexes of formula (I) or (Ia) (and in the method of the invention),
X² denotes Br, or I.

Preferably, in the complexes of formula (I) or (Ia) (and in the method of the invention),
X² denotes Br.

Preferably, in the complexes of formula (I) or (Ia) (and in the method of the invention),
X² denotes Cl.

Preferably, in the complexes of formula (I) or (Ia) (and in the method of the invention),
X¹ denotes CI and
X² denotes Cl.

Preferably, in the complexes of formula (I), (Ia), (II) and (IIa),
R^{a} denotes C₁-C₄-alkyl.

Preferably, in the complexes of formula (I), (Ia), (II) and (IIa),
R^{a} denotes methyl.

Preferably, in the complexes of formula (II) and (IIa),
L′ denotes NR′R″R′″;
wherein
R′, R″ and R′″ each independently denote C₁-C₄ alkyl, C₃-C₆ cycloalkyl or C₆-C₁₀ aryl, which may be optionally substituted by one or more R^{a}; or together two of R′ and R″ combine to form a ring containing 4-6 alkylene units; or
R′, R″ and R′″ together with the N combine to form quinuclidine (1-azabicyclo[2.2.2]octane) or DABCO (1,4-diazabicyclo[2.2.2]octane); and each R^{a} independently denotes halogen, NO₂, C₁-C₄-alkyl, or C₁-C₄-alkoxy.

Preferably, in the complexes of formula (II) and (IIa),
L′ denotes NR′R″R′″;
wherein
R′, R″ and R′″ each independently denote C₁-C₄ alkyl, C₃-C₆ cycloalkyl; or together two of R′ and R″ combine to form a ring containing 4-6 alkylene units; or
R′, R″ and R′″ together with the N combine to form quinuclidine (1-azabicyclo[2.2.2]octane) or DABCO (1,4-diazabicyclo[2.2.2]octane).

Preferably, in the complexes of formula (II) and (IIa),
L′ denotes NR′R″R′″;
wherein
R′, R″ and R′″ each independently denote C₁-C₄ alkyl; or together two of R′ and R″ combine to form a ring containing 4-5 alkylene units; or
R′, R″ and R′″ together with the N combine to form quinuclidine (1-azabicyclo[2.2.2]octane) or DABCO (1,4-diazabicyclo[2.2.2]octane).

Preferably, in the complexes of formula (II) and (IIa),
L′ denotes NR′R″R′″;
wherein
R′, R″ and R′″ each independently denote C₁-C₄ alkyl; or together two of R′ and R″ combine to form a ring containing 4-5 alkylene units; or
R′, R″ and R′″ together with the N combine to form quinuclidine (1-azabicyclo[2.2.2]octane).

Preferably, in the complexes of formula (II) and (IIa),
L′ denotes NR′R″R′″;
wherein
R′, R″ and R′″ each independently denote methyl or ethyl; or together two of R′ and R″ combine to form a ring containing 4-5 alkylene units; or
R′, R″ and R′″ together with the N combine to form quinuclidine (1-azabicyclo[2.2.2]octane).

Preferably, in the complexes of formula (II) and (IIa),
L′ denotes NR′R″R′″;
wherein
R′, R″ and R′″ each independently denote C₁-C₄ alkyl.

Preferably, in the complexes of formula (II) and (IIa),
L′ denotes triethylamine.

The above noted preferred embodiments can of course be combined with one another. Thus, particularly preferred embodiments of the invention include:

### Embodiment 1

A complex of formula (Ia)

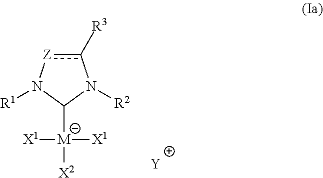

wherein
M denotes a metal selected from Pd, Pt and Ni;
X¹ denotes F, Cl, Br, I, OH, or C₁-C₄-alkoxide;
X² denotes F, Cl, Br, I, CN, SCN, NCS, N₃ or R^{x}CO₂;
R^{x} denotes H or C₁-C₄ alkyl;
R¹ and R² independently denote C₁-C₄-alkyl, C₃-C₁₀ cycloalkyl; or C₆-C₁₀ aryl optionally substituted with 1 to 5 R^{a};
Z denotes CHR⁴ or CR⁴;
R³ and R⁴ independently denote H, C₁-C₄ alkyl, C₆-C₁₀ aryl, C₃-C₆ cycloalkyl, C₁-C₄-alkyl-C₃-C₆-cycloalkyl, which may be optionally substituted by one or more R^{a}; or
R³ and R⁴ together form a 5-7 membered ring, which may be saturated or unsaturated; or
R² and R³ and/or R¹ and R⁴ may together form an alkylene group having 3 or 4 carbon atoms;
each R^{a} independently denotes halogen, NO₂, C₁-C₄-alkyl, or C₁-C₄-alkoxy;
   and
Y denotes a non-coordinating cation.

### Embodiment 2

A complex of formula (Ia)

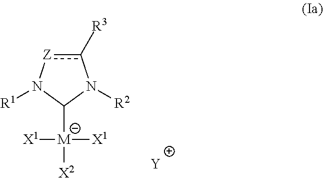

wherein
M denotes Pd;
X¹ denotes F, Cl, Br, I, or C₁-C₄-alkoxide;
X² denotes F, Cl, Br, I, CN, SCN, NCS, N₃ or R^{x}CO₂;
R^{x} denotes H or C₁-C₄ alkyl;
R¹ and R² independently denote C₁-C₄-alkyl, cyclohexyl, adamantyl; or phenyl optionally substituted with 1 to 5 R^{a};
Z denotes CHR⁴ or CR⁴;
R³ and R⁴ independently denote H, C₁-C₄ alkyl, C₁-C₄-alkyl-C₆-C₁₀-aryl, C₁-C₄-alkyl-C₃-C₆-cycloalkyl, which may be optionally substituted by one or more R^{a}; or
R³ and R⁴ may together form —(CH)₄— when Z denotes CR⁴;
each R^{a} independently denotes halogen, NO₂, C₁-C₄-alkyl, or C₁-C₄-alkoxy;
   and
Y denotes a non-coordinating cation.

### Embodiment 3

A complex of formula (Ia)

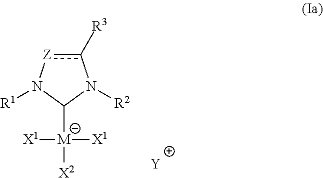

wherein
M denotes Pd;
X¹ denotes F, Cl, Br, or I;
X² denotes Cl, Br, I, or CN;
R¹ and R² independently denote C₁-C₄-alkyl, cyclohexyl, adamantyl; or phenyl optionally substituted with 1 to 5 R^{a};
Z denotes CHR⁴ or CR⁴;
R³ and R⁴ independently denote H, C₁-C₄ alkyl, C₁-C₄-alkyl-C₆-C₁₀-aryl, C₁-C₄-alkyl-C₃-C₆-cycloalkyl, which may be optionally substituted by one or more R^{a}; or
R³ and R⁴ may together form —(CH)₄— when Z denotes CR⁴;
each R^{a} independently denotes halogen, NO₂, C₁-C₄-alkyl, or C₁-C₄-alkoxy;
   and
Y denotes a non-coordinating cation.

### Embodiment 4

A complex of formula (Ia)

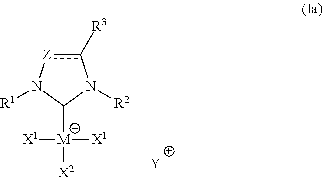

wherein
M denotes Pd;
X¹ denotes Cl, Br, or I;
X² denotes Br, or I;
R¹ and R² independently denote C₁-C₄-alkyl, cyclohexyl, adamantyl; or phenyl optionally substituted with 1 to 5 R^{a};
Z denotes CHR⁴ or CR⁴;
R³ and R⁴ independently denote H or C₁-C₄ alkyl; or
R³ and R⁴ may together form —(CH)₄— when Z denotes CR⁴;
each R^{a} independently denotes C₁-C₄-alkyl; and
Y denotes a non-coordinating cation.

As noted above, Y denotes a non-coordinating cation.

By “non-coordinating cation” is meant a cationic species that interacts weakly or not at all with anions. The non-coordinating cation within the context of the present invention can be any species that does not interact with the anionic metal complex. Thus, preferably the non-coordinating cation does not contain any nucleophilic lone pairs or nucleophilic 7-bonds, or any moieties that would be capable of coordinating to a metal ion as a ligand.

Non-coordinating cation Y is not covalently bonded to the metal complex, and is a separate chemical entity.

By “nucleophilic lone pairs” is meant lone pairs that are capable of acting as a nucleophile and coordinating a metal ion. Typically, nucleophilic lone pairs are lone pairs on N, O or S atoms.

By “nucleophilic 7-bonds” is meant double bonds which are capable of acting as a nucleophile. Typically, this does not include aromatic double bonds unless the aromatic ring is highly electron rich due to electron donating substituent groups. Thus, the non-coordinating cation may contain aromatic rings, but does not generally contain reactive ethenyl units.

It is also preferred that the non-coordinating cation does not contain any acidic protons. In the present invention, this means that preferably the non-coordinating cation does not contain any protons with a pKₐ of 12 or below, preferably 13 or below, more preferably 14 or below, more preferably 15 or below. Thus, the non-coordinating cation preferably does not contain any protons which are moderately acidic, which means that the conjugate base (which would potentially be capable of interacting with the metal complex) does not readily form.

The non-coordinating cation preferably does not contain any reactive C—H bonds, such as C—H bonds that readily undergo deuterium exchange. Thus, preferably, the non-coordinating cation does not contain any C—H bonds with a pKₐ of 25 or below, more preferably 26 or below, even more preferably 27 or below.

The “pKₐ” or acid dissociation constant is a measure of the strength of an acid in solution. As a general guide, acids will have low or even negative pKₐ values, while non-acids such as ethane will have high positive pKₐ values. The pKₐ depends on the temperature, the ionic strength and the dielectric constant of the solvent, as well as the ability of the solvent to form hydrogen bonds. Compounds can therefore have different pKₐ values in different solvents. As an example, Sowmiah et al., showed that a compound having a pKₐ of 23.0 in water has a pKₐ of 21.1 when measured in DMSO (Molecules, 2009, 14, 3780-3813).

There are many ways to measure the pKₐ of a molecule, including potentiometric titration, NMR spectroscopy, UV/vis spectroscopy, polarimetry, conductometry, fluorometry, HPLC, electrophoresis and calorimetry. More recently, computational methods have been developed. The choice of method will depend on the molecule being investigated and the degree of accuracy required from the method. A review of the various methods available is provided in Reijenga et al., Analytical Chemistry Insights, 2013, 8, 53-71. Potentiometric titration or UV/vis spectroscopy are preferred, with potentiometric titration being the most preferred.

Regardless of the method used to measure or predict the pKₐ, the outcomes should be identical (allowing for experimental error) provided that the temperature, the ionic strength and the solvent are kept constant.

For the avoidance of doubt, where pKₐ values are quoted herein, these are the pKₐ values of the molecule in water at 25° C.

Any C—H bonds with pKₐ values of around 25 or below in the non-coordinating may be capable of reacting with the metal centre in the compound of formula (I). For example, when the metal is palladium or nickel, it can insert into reactive C—H bonds even under moderate conditions, as shown in Lebel et al., J. Amer. Chem. Soc. 2004, 126, 5046-5047, Clement et al., Angew. Chem. Int. Ed. 2004, 43, 1277-1279 and Grundemann et al., J. Chem. Soc., Dalton Trans., 2002, 2163-2167. These reactions are known to modify the structure and the electronic properties of the metal centre and therefore the catalytic behaviour leading to decreased reactivity and/or unexpected side reactions and products. Avoiding any reactive C—H bonds in the non-coordinating cation mitigates the risk of such reactions taking place.

An example of a C—H bond having a relatively low pKₐ is the C—H bond that may be found between the two nitrogen atoms of an N-heterocyclic structure, such as that drawn below:

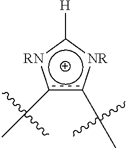

wherein each R may independently be alkyl or aryl, the dotted line may be a double bond. The two bonds intersected with wavy lines may be e.g. alkyl or aryl, or may join together to for a saturated or unsaturated ring.

The C—H bonds between the two nitrogen atoms of the N-heterocyclic structure drawn above have a pKₐ of about 21-24. In contrast, the C—H bonds on an alkane have a pKₐ of about 50.

Thus, it is preferred that the non-coordinating cation does not interact with the metal centre of the complex of formula (II) (or (IIa)) or the complex of formula (I) (or (Ia)).

In some embodiments, the non-coordinating cation preferably does not comprise a imidazolium, a tetrahydroimidazolium, or a triazolium moiety (i.e. a cation that comprises a 5-membered heterocyclic ring containing a positive charge, which can be deprotonated to form an imidazol-2-ylidene, a tetrahydroimidazol-2-ylidene, or a triazol-5-ylidene).

Preferably, in the non-coordinating cation the positive charge is located on a nitrogen or phosphorus atom. More preferably, the positive charge is located on a nitrogen atom. For example, Y may be a quaternary ammonium compound, or a pyridinium cation.

In some aspects of the invention, Y is only present to ensure charge neutrality.

In other aspects, Y may have an active role in the catalytic reactions carried out by the complex of formula (I). For example, Y may be a chiral cation. Reactions where cationic chiral auxiliaries have been used in enantioselective synthesis are known in the art, for example from Chem. Rev., 2003, 103, 3013-3028; and J. Org. Chem., 2011, 76, 4337-4357.

Thus, in some aspects, Y is a chiral cation.

Examples of chiral non coordinating cations are show below:

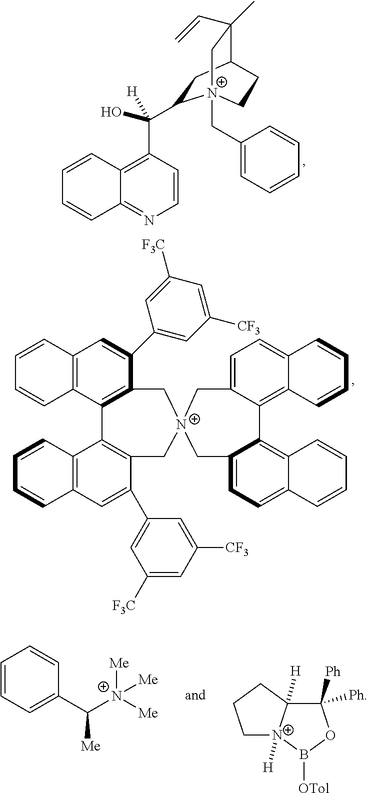

Phosphorus based chiral non coordinating cations include the following:

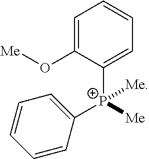

Y may also act as a co-catalyst, for example N-heterocylic carbene moieties are known to be active in catalysis when not coordinated to metal centres (See Chem. Rev., 2007, 107, 5606-5655; Angew. Chem. Int. Ed., 2007, 46, 2988-3000; and Acc. Chem. Res., 2004, 37, 534-541). Precursors of such moieties such as imidizolium salts have therefore been used in combination with other catalysts such as palladium complexes in multistep syntheses, whereby the N-heterocyclic carbene and metal complex catalyse separate steps.

Thus, in some aspects, Y is an imidazolium cation.

Preferably, in the complexes of formula (I), (Ia), (II) and (IIa) and in Embodiments 1-4 Y is selected from:

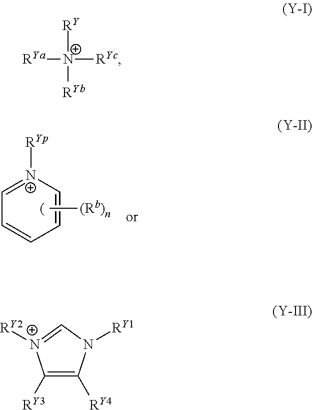

wherein

R^{Y}, R^{Ya}, R^{Yb} and R^{Yc} each independently denote alkyl, cycloalkyl, alkylcycloalkyl, aryl or alkylaryl, which may be optionally substituted by one or more R^{b}; or

together two of R^{Ya} and R^{Yb} together with the N combine to form a ring containing 4-6 alkylene units; or

R^{Ya}, R^{Yb} and R^{Yc} together with the N combine to form quinuclidine (1-azabicyclo[2.2.2]octane);

R^{Yp} denotes alkyl, cycloalkyl, alkylcycloalkyl, aryl or alkylaryl, which may be optionally substituted by one or more R^{b};

R^{Y1} and R^{Y2} independently denote C₁-C₄-alkyl, C₃-C₁₀ cycloalkyl; or C₆-C₁₀ aryl optionally substituted with 1 to 5 R^{b};

R^{Y3} and R^{Y4} independently denote H, alkyl, aryl, cycloalkyl, alkylaryl, or alkylcycloalkyl, which may be optionally substituted by one or more R^{b}; or

R^{Y3} and R^{Y4} together form —(CH)₄—; or

R^{Y2} and R^{Y3} and/or R^{Y1} and R^{Y4} may together form an alkylene group having 3 or 4 carbon atoms;

each R^{b} independently denotes halogen, NO₂, C₁-C₄-alkyl, or C₁-C₄-alkoxy; and

n denotes and integer from 0 to 5.

Preferably, in the complexes of formula (I), (Ia), (II), (IIa) and in Embodiments 1-4,

R¹ and R² are the same.

Preferably, in the complexes of formula (I), (Ia), (II), (IIa), R¹ and R² are the same and are both C₆-C₁₀ aryl optionally substituted with 1 to 5 R^{a}. More preferably, said C₆-C₁₀ aryl group is a phenyl ring.

Preferably, in the complexes of formula (I), (Ia), (II), (IIa) and in Embodiments 1-4, R¹ and R² are phenyl substituted with C₁-C₄-alkyl. More preferably, said C₁-C₄-alkyl is C₃ alkyl. Most preferably, said C₃ alkyl is isopropyl.

Preferably, in the complexes of formula (I), (Ia), (II) and (IIa) and in Embodiments 1-4:

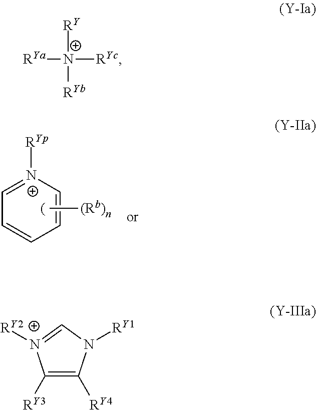

wherein

R^{Y}, R^{Ya}, R^{Yb} and R^{Yc} each independently denote C₁-C₄ alkyl, C₃-C₆ cycloalkyl, C₁-C₄-alkyl-C₃-C₆-cycloalkyl, C₆-C₁₀ aryl or C₁-C₄-alkyl-C₆-C₁₀-aryl, which may be optionally substituted by one or more R^{b}; or

together two of R^{Ya} and R^{Yb} together with the N combine to form a ring containing 4-6 alkylene units; or

R^{Ya}, R^{Yb} and R^{Yc} together with the N combine to form quinuclidine (1-azabicyclo[2.2.2]octane);

R^{Yp} denotes C₁-C₄ alkyl, C₃-C₆ cycloalkyl, C₁-C₄-alkyl-C₃-C₆-cycloalkyl, C₆-C₁₀ aryl or C₁-C₄-alkyl-C₆-C₁₀-aryl, which may be optionally substituted by one or more R^{b};

R^{Y1} and R^{Y2} independently denote C₁-C₄-alkyl, C₃-C₁₀ cycloalkyl; or C₆-C₁₀ aryl optionally substituted with 1 to 5 R^{b};

R^{Y3} and R^{Y4} independently denote H, C₁-C₄ alkyl, C₆-C₁₀ aryl, C₃-C₆ cycloalkyl, C₁-C₄-alkyl-C₆-C₁₀-aryl, or C₁-C₄-alkyl-C₃-C₆-cycloalkyl, which may be optionally substituted by one or more R^{b}; or

R^{Y3} and R^{Y4} together form —(CH)₄—; or

R^{Y2} and R^{Y3} and/or R^{Y1} and R^{Y4} may together form an alkylene group having 3 or 4 carbon atoms;

each R^{b} independently denotes halogen, NO₂, C₁-C₄-alkyl, or C₁-C₄-alkoxy; and

n denotes and integer from 0 to 5.

Preferably, in the complexes of formula (I), (Ia), (II) and (IIa) and in Embodiments 1-4,

Y has formula (Y-I) or (Y-II).

Preferably, in the complexes of formula (I), (Ia), (II) and (IIa) and in Embodiments 1-4,

Y has formula (Y-Ia) or (Y-IIa).

Preferably, in the complexes of formula (I), (Ia), (II) and (IIa) and in Embodiments 1-4,

Y has formula (Y-I).

Preferably, in the complexes of formula (I), (Ia), (II) and (IIa) and in Embodiments 1-4,

Y has formula (Y-Ia).

Preferably, in the complexes of formula (I), (Ia), (II) and (IIa) and in Embodiments 1-4,

Y has formula (Y-III).

Preferably, in the complexes of formula (I), (Ia), (II) and (IIa) and in Embodiments 1-4,

Y has formula (Y-IIIa).

Preferably, in the cations (Y-I), (Y-Ia), (Y-II), (Y-IIa), (Y-III), and (Y-IIIa),

R^{b} denotes C₁-C₄ alkyl.

Preferably, in the cations (Y-I) and (Y-Ia), R^{b} denotes C₁-C₄ alkyl.

Preferably, in the cations (Y-I) and (Y-Ia), R^{b} denotes C₄ alkyl. More preferably, in the cations (Y-I) and (Y-Ia), R^{b} denotes n-butyl, i-butyl, t-butyl or s-butyl. Most preferably, in the cations (Y-I) and (Y-Ia), R^{b} denotes n-butyl.

Preferably, in the cations (Y-I), (Y-Ia), (Y-II), (Y-IIa), (Y-III), and (Y-IIIa),

R^{Y}, R^{Ya}, R^{Yb} and R^{Yc} each independently denote C₁-C₄ alkyl, C₃-C₆ cycloalkyl, C₁-C₄-alkyl-C₃-C₆-cycloalkyl, or C₁-C₄-alkyl-C₆-C₁₀-aryl, which may be optionally substituted by one or more R^{b}; or

together two of R^{Ya} and R^{Yb} together with the N combine to form a ring containing 4-5 alkylene units; or

R^{Ya}, R^{Yb} and R^{Yc} together with the N combine to form quinuclidine (1-azabicyclo[2.2.2]octane);

R^{Yp} denotes C₁-C₄ alkyl, C₃-C₆ cycloalkyl, C₆-C₁₀ aryl or C₁-C₄-alkyl-C₆-C₁₀-aryl, which may be optionally substituted by one or more R^{b};

R^{Y1} and R^{Y2} independently denote C₁-C₄-alkyl, cyclohexyl, adamantyl; or phenyl optionally substituted with 1 to 5 R^{b};

R^{Y3} and R^{Y4} independently denote H or C₁-C₄ alkyl; or

R^{Y3} and R^{Y4} together form —(CH)₄—; and

R^{b} denotes C₁-C₄ alkyl.

As used herein, “alkyl” is intended to include both branched and straight-chain saturated aliphatic hydrocarbon groups having the specified number of carbon atoms. Preferred are C₁-C₆ alkyl groups. “C₁-C₆ alkyl” is intended to include C₁, C₂, C₃, C₄, C₅ and C₆ alkyl groups. Examples of alkyl include, but are not limited to, methyl, ethyl, n-propyl, i-propyl, n-butyl, i-butyl, sec-butyl, t-butyl, n-pentyl, n-hexyl, 2-methylbutyl, 2-methylpentyl, 2-ethylbutyl, 3-methylpentyl, and 4-methylpentyl.

The term “cycloalkyl” refers to cyclized alkyl groups, including mono-, bi- or poly-cyclic ring systems. Preferred are C₁-C₆ cycloalkyl groups. “C₁-C₆ cycloalkyl” is intended to include C₁, C₂, C₃, C₄, C₅ and C₆ alkyl groups. Example cycloalkyl groups include, but are not limited to, cyclopropyl, cyclobutyl, cyclopentyl, cyclohexyl, norbornyl, adamantyl and the like. Branched cycloalkyl groups such as 1-methylcyclopropyl and 2-methylcyclopropyl are included in the definition of “cycloalkyl”.

“Halogen” as used herein refers to fluoro, chloro, bromo, and iodo.

“Alkoxy” represents an alkyl group as defined above with the indicated number of carbon atoms attached through an oxygen bridge. For example, “C₁-C₄-alkoxy” is intended to include C₁, C₂, C₃ and C₄ alkoxy groups. Examples of alkoxy include, but are not limited to, methoxy, ethoxy, n-propoxy, i-propoxy, n-butoxy, s-butoxy, t-butoxy, n-pentoxy, and s-pentoxy.

As used herein, the term “aryl”, is intended to mean an aromatic moiety containing, if specified, the specified number of carbon atoms. Preferred are C₆-C₁₀ aryl groups. Examples of aryl include, but are not limited to phenyl and naphthyl.

Preferred complexes of formula (I) are shown below:

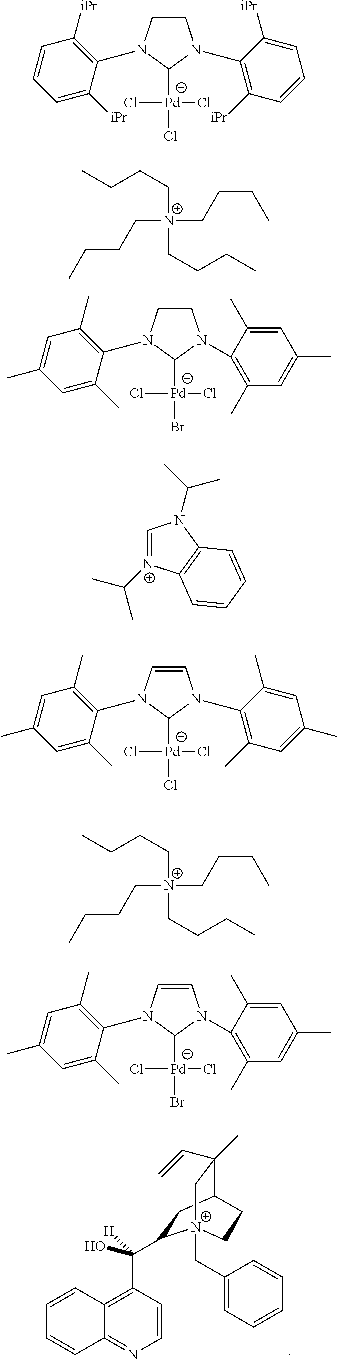

The complexes of formula (I) are active as catalysts in a number of reactions, in particular carbon-carbon and carbon-heteroatom (e.g. carbon-oxygen) bond-forming reactions (through C—H or C-halogen activation), tandem organic/organometallic catalysis and enantioselective catalysis.

Palladium is very versatile and can be used in a number carbon-carbon and carbon-heteroatom bond-forming reactions, such as the “Heck Reaction” (also called the Mizoroki-Heck reaction) between alkenes and aryl halides; the “Suzuki Reaction” (also known as the Suzuki-Miyaura reaction) between aryl halides and boronic acids; the “Stille Reaction” between organohalides and organo tin compounds, the “Buchwald-Hartwig Reaction” between an aryl halide and an amine and the “Negishi Reaction” between an organohalide and an organozinc compound. These reactions are now very common in synthetic organic chemistry and there has been interest in improving yields and ease of recovering the product and/or catalyst.

A typical reaction scheme for a palladium catalysed C—H activation reaction is shown below (Chen et al., Angew. Chem. Int. Ed., 2009, 48(28), 5094-5115):

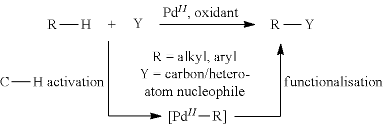

Palladium catalysed C—H activation can lead to the formation of carbon-carbon bonds (Chiong et al., J. Am. Chem. Soc., 2007, 129, 9879-9884):

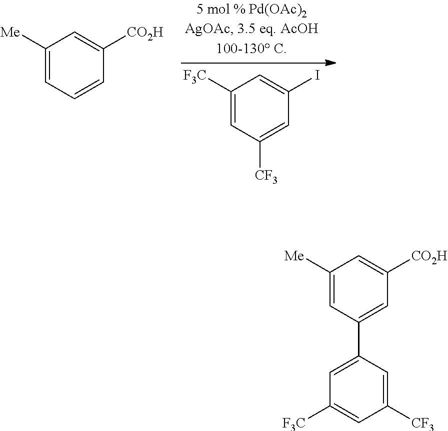 [SMILES: Cc1cccc(C(=O)O)c1 ; FC(F)(F)c1cc(I)cc(C(F)(F)F)c1 ; Cc1cc(C(=O)O)cc(-c2cc(C(F)(F)F)cc(C(F)(F)F)c2)c1]

Palladium catalysed C—H activation can also lead to the formation of carbon-halide or carbon-oxygen bonds (Stowers and Sandford, Org. Lett., 2009, 11(20), 4584-4587):

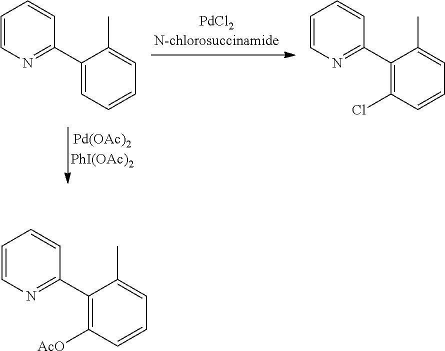 [SMILES: Cc1ccccc1-c1ccccn1 ; Cc1cccc(Cl)c1-c1ccccn1 ; CC(=O)Oc1cccc(C)c1-c1ccccn1]

As noted previously, many of the complexes known in the art catalyse carbon-carbon bond-forming reactions using transitions between the M⁰ and M^{II} oxidation states, where M is the metal ion at the core of the complex. Arylation of sp² and sp^{a} hybridised C—H bonds via the Pd^{II}/Pd^{IV} catalytic cycle is less well characterised and few examples are known. This reaction was first reported by Tremont and Rhaman, who observed methylation of ortho C—H bonds in aniline (J. Am. Chem. Soc., 1984, 106, 5759). A proposed catalytic cycle for this reaction is shown below:

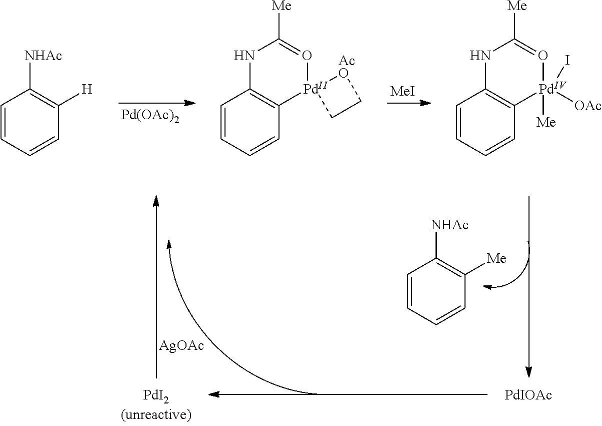

Other examples of C—H arylation via the Pd^{II}/Pd^{IV} catalytic cycle use hypervalent iodine compounds (see Xia and Chen, Synth. Commun., 2000, 30, 531). It is believed that hypervalent iodine compound plays a similar role to Mel in the catalytic cycle outlined above:

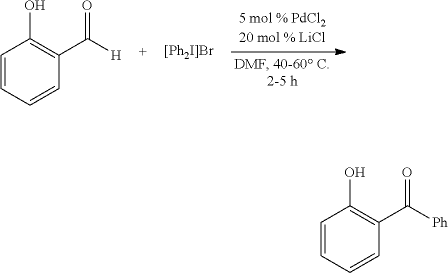

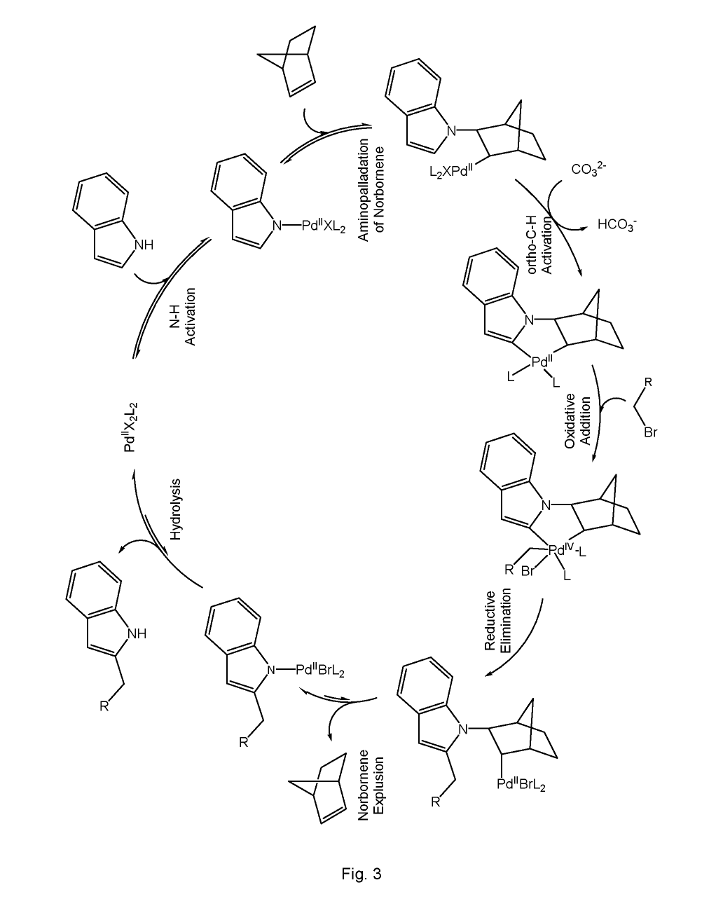

It has also been found that the Pd^{II}/Pd^{IV} catalytic cycle may be involved in palladium catalysed 2-alkylation of indoles using norbornene (J. Am. Chem. Soc., 2012, 134, 14563-14572). A proposed catalytic cycle for this reaction is shown in FIG. 3.

Palladium complexes have also been used in the enantioselective hydrogenation of enones (Tsuchiya et al., Org. Lett., 2006, 8(21), 4851-4854):

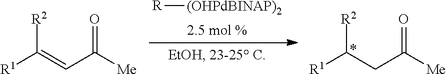

Palladium can also be used in tandem catalytic processes. Tandem catalysis is a technique that uses multiple catalysts on a single molecule in single reaction step to produce a product otherwise not accessible by a single catalyst. Tandem benzoin/allylation processes are explored in Lebeuf et al., Org. Lett, 2008, 10(19), 4243-4246. One example of a tandem catalytic process is shown below:

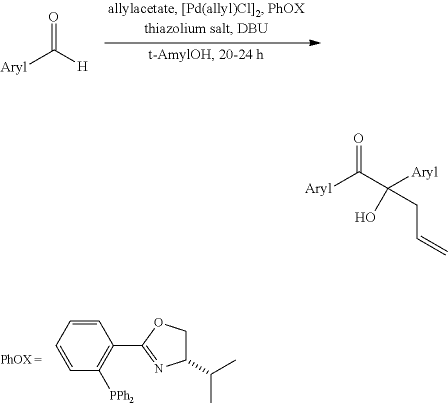

Platinum has also been used as a catalyst in carbon-carbon bond forming reactions. Platinum catalysts for Suzuki biaryl coupling reactions are discussed in Bedford et al. (Organometallics, 2002, 21(13), 2599-2600):

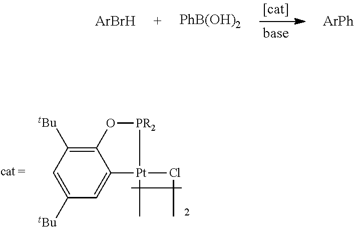

Platinum catalysed Mizoroki-Heck reactions are also known (see The Mizoroki-Heck Reaction, Wiley, 2009, Edited by Martin Oestrich):

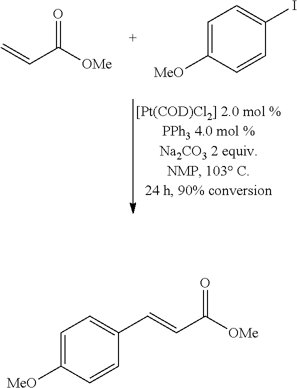

In transition-metal-catalyzed cross-coupling reactions, the use of the first row transition metals as catalysts is more appealing than use of precious metal catalysts owing to their significantly lower cost. Recent efforts have therefore focused on use of nickel in reactions commonly catalysed by the precious metals palladium or platinum. It has been found that nickel can catalyse Suzuki-Miyaura couplings. Notably, a broad range of aryl electrophiles such as phenols, aryl ethers, esters, carbonates, carbamates, sulfamates, phosphates, phosphoramides, phosphonium salts, and fluorides, as well as various alkyl electrophiles can be coupled efficiently with boron reagents in the presence of nickel catalysts (see Han Chem. Soc. Rev., 2013, 42, 5270-5298).

Achiral nickel catalysts have also been used in Negishi-type reactions and stereospecific cross coupling reactions of secondary benzylic esters has been reported (see Wisniewska et al. J. Am. Chem. Soc., 2013, 135 (24), 9083-9090):

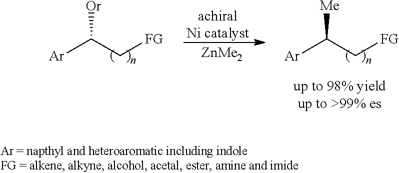

Nickel catalysed decarbonylative C—H coupling reactions have also been recently reported by Correa et al. (Angewante Chemie, 2013, 52(7), 1878-1880):

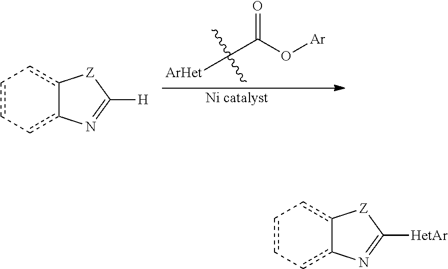

The complexes of formula (I) have catalytic activity and can be used in reactions such as those described above. The complexes are particularly advantageous as they are relatively stable in air and non-anhydrous environments. Moreover, the complexes are relatively electron rich due to the X² ligand, rendering them particularly suitable for reactions which typically invoke the M^{IV} oxidation state, such as C—H bond activation.

Thus, the present invention also relates to the use of a complex of formula (I) or (Ia) as a catalyst, preferably as a homogeneous catalyst in a reaction involving an organic compound. Preferably, the reaction takes place in a solvent.

Preferably, the present invention relates to the use of a complex of formula (I) or (Ia) as a catalyst for a Heck reaction, or a derivative reaction thereof.

Preferably, the present invention relates to the use of a complex of formula (I) or (Ia) as a catalyst for a Suzuki reaction, or a derivative reaction thereof.

Preferably, the present invention relates to the use of a complex of formula (I) or (Ia) as a catalyst for a Stille reaction, or a derivative reaction thereof.

Preferably, the present invention relates to the use of a complex of formula (I) or (Ia) as a catalyst for a Buchwald-Hartwig reaction, or a derivative reaction thereof.

Preferably, the present invention relates to the use of a complex of formula (I) or (Ia) as a catalyst for a Negishi reaction, or a derivative reaction thereof.

Preferably, the present invention relates to the use of a complex of formula (I) or (Ia) as a catalyst for the formation of a carbon-carbon bond, more preferably wherein one or both of the carbon atoms is sp² hybridised.

Preferably, the present invention relates to the use of a complex of formula (I) or (Ia) as a catalyst for C—H bond activation.

Preferably, the present invention relates to the use of a complex of formula (I) or (Ia) as a catalyst for activation of the C—H bond between the two nitrogen atoms of an N-heterocyclic carbene.

Preferably, the present invention relates to the use of a complex of formula (I) or (Ia) as a catalyst for activation of a C—H bond leading to the formation of a new carbon-carbon bond.

Preferably, the present invention relates to the use of a complex of formula (I) or (Ia) as a catalyst for activation of a C—H bond leading to the formation of a new carbon-halide bond.

Preferably, the present invention relates to the use of a complex of formula (I) or (Ia) as a catalyst for activation of a C—H bond leading to the formation of a new carbon-oxygen bond.

In the above embodiments, it is preferred that the C—H bond to be activated is on an aromatic compound.

Preferably, the present invention relates to the use of a complex of formula (I) or (Ia) as a catalyst for oxidation of an alcohol group to a ketone, aldehyde or carboxylic acid functional group.

Preferably, the present invention relates to the use of a complex of formula (I) or (Ia) as a catalyst for a carbon-carbon bond forming reaction via the M^{II} and M^{IV} oxidation states.

Preferably, the present invention relates to the use of a complex of formula (I) or (Ia) as a catalyst for a carbon-carbon bond forming reaction via the Pd^{II} and Pd^{IV} oxidation states.

Preferably, the present invention relates to the use of a complex of formula (I) or (Ia) as a catalyst for a 2-alkylation of indoles.

More preferably, the present invention relates to the use of a complex of formula (I) or (Ia) as a catalyst for a 2-alkylation of indoles in the presence of norbornene.

Preferably, the present invention relates to the use of a complex of formula (I) or (Ia) as a catalyst for a hydrogenation reaction, preferably hydrogenation of an olefin.

Preferably, the present invention relates to the use of a complex of formula (I) or (Ia) as a catalyst for an enantioselective reaction.

Preferably, the present invention relates to the use of a complex of formula (I) or (Ia) as a catalyst for the enantioselective hydrogenation of enones.

Preferably, the present invention relates to the use of a complex of formula (I) or (Ia) in a reaction catalysed by a tandem organic/organometallic catalyst, wherein the complex of formula (I) or (Ia) forms at least the organometallic catalyst in the tandem catalyst.

In the above embodiments, in the complex of formula (I) or (Ia) for use as a catalyst, M is Pd or Pt. Preferably, M is Pd.

The present invention also relates to a process for C—H bond activation characterised in that the complex of formula (I) or (Ia) is used as a catalyst.

Preferably, the present invention relates to a process for forming a carbon-carbon bond, characterised in that the of a complex of formula (I) or (Ia) is used as a catalyst, preferably wherein one or both of the carbon atoms is sp² hybridised.

Preferably, the present invention relates to a process for activation of the C—H bond between the two nitrogen atoms of an N-heterocyclic carbene characterised in that the complex of formula (I) or (Ia) is used as a catalyst.

Preferably, the present invention relates to a process for activation of a C—H bond leading to the formation of a new carbon-carbon bond, characterised in that the complex of formula (I) or (Ia) is used as a catalyst.

Preferably, the present invention relates to a process for activation of a C—H bond leading to the formation of a new carbon-halide bond, characterised in that the complex of formula (I) or (Ia) is used as a catalyst.

Preferably, the present invention relates to a process for activation of a C—H bond leading to the formation of a new carbon-oxygen bond, characterised in that the complex of formula (I) or (Ia) is used as a catalyst.

In the above embodiments, it is preferred that the C—H bond to be activated is on an aromatic compound.

Preferably, the present invention relates to a process for forming a carbon-carbon bond via the M^{II} and M^{IV} oxidation states, characterised in that the complex of formula (I) or (Ia) is used as a catalyst.

Preferably, the present invention relates to a process for forming a carbon-carbon bond reaction via the Pd^{II} and Pd^{IV} oxidation states, characterised in that the complex of formula (I) or (Ia) is used as a catalyst.

Preferably, the present invention relates to a process for 2-alkylation of indoles, characterised in that the complex of formula (I) or (Ia) is used as a catalyst.

More preferably, the present invention relates a process for 2-alkylation of indoles in the presence of norbornene, characterised in that the complex of formula (I) or (Ia) is used as a catalyst.

Preferably, the present invention relates to a process for activation of a C—X bond leading to the formation of a new carbon-nitrogen bond, wherein X is a halide and wherein the complex of formula (I) or (Ia) is used as a catalyst.

Preferably, the present invention relates to a process for hydrogenating an olefin, characterised in that the complex of formula (I) or (Ia) is used as a catalyst.

Preferably, the present invention relates to a process for introducing enantioselectivity into a reaction, characterised in that the complex of formula (I) or (Ia) is used as a catalyst.

More preferably, the present invention relates to a process for introducing enantioselectivity into the hydrogenation of enones, characterised in that the complex of formula (I) or (Ia) is used as a catalyst.

Preferably, the present invention relates to a process using a tandem organic/organometallic catalyst, characterised in that the complex of formula (I) or (Ia) forms at least the organometallic catalyst in the tandem catalyst.

In the above processes, in the complex of formula (I) or (Ia), M is Pd or Pt. Preferably, M is Pd.

It is to be understood that where the preferred embodiments mentioned above are not mutually exclusive, they can be combined with one another. The skilled person would understand which embodiments where mutually exclusive and would thus readily be able to determine the combinations of preferred embodiments that are contemplated by the present application.

The solvents used for the catalytic processes of the invention are generally the same as the preferred solvents listed above for the method of the invention. However, often the solvents chosen for the method of the invention will precipitate the complex of the invention, making it easier to isolate. Clearly, when used as a catalyst, a different blend of solvents should be used to ensure that the catalyst remains soluble during the catalytic process.

Preferred solvents for use in the catalytic processes include 1,4-dioxane, tetrahydrofuran, acetone, C₁-C₁₀ alcohols, ethers, such as dimethyl ether and diethyl ether, benzene, toluene, xylene, mesitylene, dimethylformamide, dimethoxyethane, acetonitrile and dimethylsulfoxide.

Particularly preferred solvents include 1,4-dioxane, tetrahydrofuran, and acetone with acetone being particularly preferred. Mixtures of these solvents and water are also preferred.

Other particularly preferred solvents include alcohols such as C₁-C₁₀ alcohols. Examples of such alcohols include, but are not limited to, methanol, ethanol, n-propanol, iso-propanol, n-butanol, sec-butanol, iso-butanol, tert-butanol, pentanol, hexanol, heptanol, octanol, nonenol and decanol. Mixtures of alcohols may be used. Preferred alcohols are selected from methanol, ethanol and iso-propanol. Where alcohols are used as the solvent, they may be used alone, or as mixtures with other solvents, such as ethers or water.

Other preferred aprotic solvents for use in the catalytic processes of the invention include ethers, such as dimethyl ether and diethyl ether, benzene, toluene, xylene, mesitylene, dimethylformamide, dimethoxyethane, acetonitrile and dimethylsulfoxide. Toluene, dimethyl ether, dimethylformamide, acetonitrile and dimethylsulfoxide are particularly preferred.

The catalytic processes can often be carried out at room temperature. However, it is sometimes preferred to apply heating to ensure the reaction happens in a reasonable timeframe.

Typical temperatures for the catalytic processes include from 20° C. to 200° C., preferably from 30° C. to 150° C.

Preferred moderate temperatures for the catalytic processes include from 20° C. to 70° C., more preferably from 30° C. to 65° C., more preferably from 40° C. to 60° C.

In some circumstances, it may be necessary or desirable to heat the catalytic reaction more strongly, for example, temperatures of up to about 200° C. may be required. Preferred higher temperatures for the catalytic processes include from 70° C. to 200° C., more preferably from 100° C. to 180° C., more preferably from 120° C. to 150° C.

Not all of the solvents listed above will be compatible with all the possible catalytic reactions using the complexes of the invention. However, the skilled person would be able to select suitable solvents using the teaching presented herein and their common general knowledge in the art.

Similarly, the skilled person would appreciate that, depending on the catalytic reaction to be carried out, further additives may be desirable in the reaction mixtures, such as acids, bases or salts. Preferred bases include alkali metal or alkali earth carbonates or hydrogen carbonates, potassium or sodium tert-butoxide, and sodium or potassium hydroxide.

Catalytic reactions using the complexes of the invention may be carried out in an inert atmosphere or in air. Reactions carried out in an “inert atmosphere” are generally carried out under a blanket of inert gas, such as nitrogen or argon. Advantageously, the complexes of the invention can be used in catalytic reactions that take place in air. As the skilled person will readily appreciate, this has significant process advantages, and makes the complexes of the invention highly suitable for use in large scale industrial processes. Thus, in a preferred embodiment, catalytic reactions using the complexes of the invention take place in air.

While performing the catalytic processes of the invention, protecting groups may be present in the reagents which are used. The use of protecting groups is well known in the art (see for example, T. W. Greene and P. G. M. Wuts, Protective Groups in Organic Synthesis, 3rd Edn., John Wiley & Sons). The skilled person will be aware of particular groups available for protecting amine, amide, carboxylic acid and alcohol groups, and the conditions under which protection and deprotection can occur. Any suitable protecting groups may be present in the complexes of the invention, either to aid in the synthesis of the complexes of formula (I), or to prevent unwanted side reactions occurring.

Suitable protecting groups for an amine include carbobenzyloxy (Cbz), p-methoxybenzyl carbonyl (Moz or MeOZ), tert-butyloxycarbonyl (BOC), 9-fluorenylmethyloxycarbonyl (FMOC), acetyl (Ac), benzoyl (Bz), benzyl (Bn) group, p-methoxybenzyl (PMB), 3,4-dimethoxybenzyl (DMPM), p-methoxyphenyl (PMP) group, tosyl (Ts), nosyl (Ns) and other sulfonamides.

Suitable protecting groups for a carboxylic acid include benzyl esters, silyl esters, orthoesters and oxazoline.

Suitable protecting groups for an alcohol include acetyl (Ac) benzoyl, benzyl (Bn), β-methoxyethoxymethyl ether (MEM), [bis-(4-methoxyphenyl)phenylmethyl] (DMT), methoxymethyl ether (MOM), monomethoxytrityl (MMT), p-methoxybenzyl ether (PMB), methylthiomethyl ether, pivaloyl (Piv), tetrahydropyranyl (THP), trityl (triphenylmethyl, Tr), silyl ethers such as trimethylsilyl (TMS), tert-butyldimethylsilyl (TBDMS), tert-butyldimethylsilyloxymethyl (TOM), and triisopropylsilyl (TIPS) ethers), methyl ethers and ethoxyethyl ethers (EE).

The following Examples are given by way of illustration only in which the Figures referred to are as follows:

FIG. 1—Single molecule structure for the complex formed in Synthesis Example 1. Hydrogens removed for clarity except those on the backbone of the N-heterocyclic carbene and the closest to the palladate from the benzimidazole.

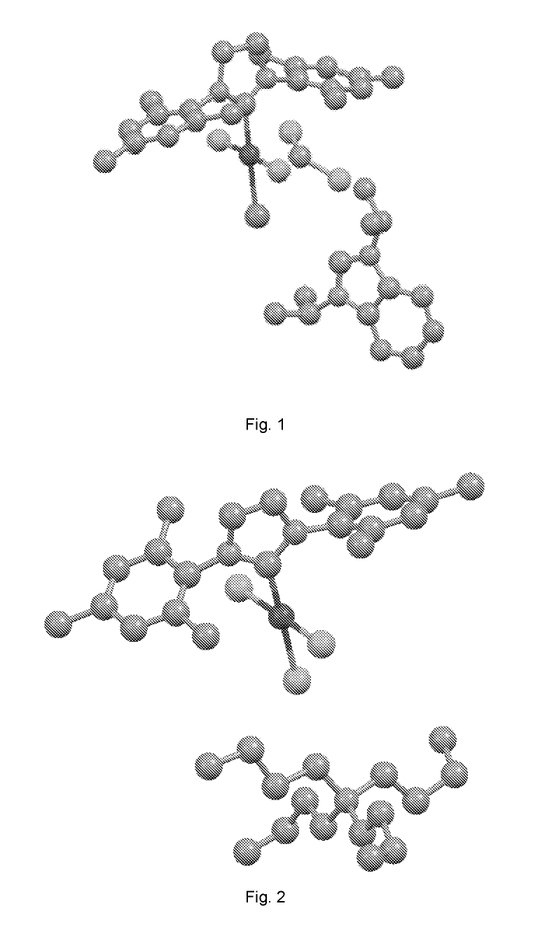

FIG. 2—Single molecule structure for the complex formed in Synthesis Example 2. Hydrogens removed for clarity except those on the backbone of the NHC and a significant H in the tetra-n-butylammonium.

FIG. 3—Reaction scheme utilising the Pd^{II} and Pd^{IV} oxidation states.

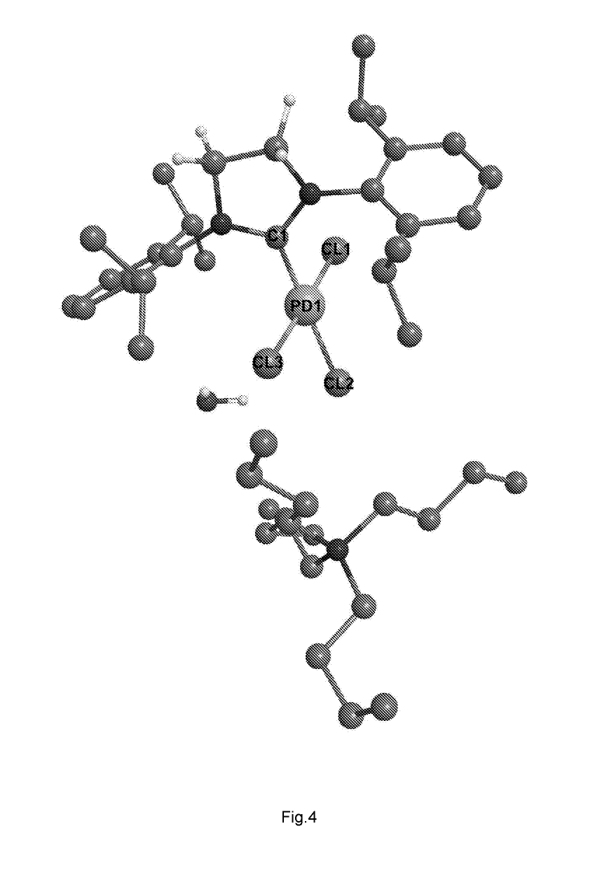

FIG. 4—Single molecule structure for the complex formed in Synthesis Example 3, co-crystallized with a molecule of water. Hydrogens removed for clarity except those on the backbone of the imidazolidine ring.

### SIMes=

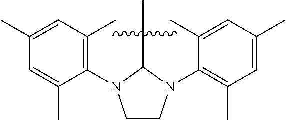

### IMES=

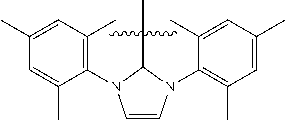

### TEA=Triethylamine SYNTHESIS EXAMPLE 1

### [(SIMes)PdCl₂Br]⁻ [1,3-Diisopropylbenzimidazolium]⁺

In a glove box, (SIMes)PdCl₂(TEA) (0.585 g, 1 mmol) and 1,3-diisopropylbenzimidazolium (0.31 g 1.1 mmol) were added in turn to vial equipped with a magnetic stirrer and sealed with a crew cap fitted with a septum. The sealed vial was then taken out of the glovebox and 5 ml of dry THF were syringed in. The solution was left to stir overnight. The next day the pale yellow precipitate which had formed was removed via vacuum filtration and identified by ¹H NMR and single crystal X-ray diffraction (the complex crystallized with one molecule of DCM, see FIG. 1, all hydrogen atoms removed for clarity) to be the titled complex [(SIMes)PdCl₂Br]⁻ [1,3-diisopropylbenzimidazolium]⁺ (0.75 g 98% yield).

¹H NMR (500 MHz, CDCl₃) δ: 11.314 (s, 1H), 7.728-7.801 (m, 2H), 7.563-7.635 (m, 2H), 6.901-7.008 (m, 4H), 5.095-5.203 (sep, 2H) 3.694-3.977 (m, 4H) 2.208-2.612 (m, 12H) 1.732-1.815 (d, 12H).

### SYNTHESIS EXAMPLE 2

### [(IMes)PdCl₃]⁻ [tetra-n-butylammonium]⁺

In a glove box, (IMes)PdCl₂(TEA) (100 mg, 0.17 mmol) and tetra-n-butylammonium chloride (53 mg, 0.19 mmol) were added in turn to vial equipped with a magnetic stirrer and sealed with a crew cap fitted with a septum. The sealed vial was then taken out of the glovebox and 2 mL of dry acetone was syringed in. The solution was allowed to stir overnight, and then the solvent was removed under vacuum. The residue was washed with diethyl ether and filtered, yielding the title compound as a pale yellow solid (79 mg).

¹H NMR (500 MHz, CDCl₃) δ: 6.919-7.080 (m, 6H) 3.228-3.312 (m, 8H), 1.860-2.531 (m, 18H), 1.548-1.649 (m, 8H), 1.356-1.452 (app sextet 8H), 0.932-1.014 (t, 12H).

FIG. 2 shows a crystal structure for the complex (all hydrogen atoms removed for clarity).

### SYNTHESIS EXAMPLE 3

### Synthesis of [(SIPr)PdCl₃]⁻ [tetra-n-butylammonium]⁺

(2): A reaction vial was loaded with a magnetic stirring bar, (SIPr)PdCl₂(TEA) (502 mg, 0.75 mmol), tetrabutylammonium chloride (231 g, 0.83 mmol) and 2 mL of dry acetone. The solution was allowed to stir at room temperature overnight. Removal of the solvent in vacuo afforded a yellow oil, which was dissolved in ethyl acetate and triturated with hexane to yield the title compound as a pale yellow solid (613 mg, 97%).

¹H NMR (500 MHz, C₆D₆); δ 7.285-7.202 (m, 6H), 3.881-3.792 (m, 4H), 3.547 (s 4H), 3.157-3.088 (m, 8H), 1.765 (d, J=6.5 Hz, 12H), 1.462-1.370 (m 8H), 1.334-1.250 (m 8H), 1.205 (d, J=6.5 Hz 12H), 0.95 (t, J=7.3 Hz 12H).

13C {1H} NMR (100 MHz, C6D6): δ 190.4, 147.6, 136.5, 128.5, 124.1, 58.3, 53.3, 28.6, 26.6, 24.7, 24.0, 19.7, 13.9.

Anal. Calcd for C₄₃H₇₅Cl₃N₃Pd: C, 61.06; H, 8.82; N, 4.97. Found: C, 60.94; H, 8.77; N, 4.94.

### CATALYTIC EXAMPLE 1

Complexes of the invention can be used in palladium catalysed anaerobic oxidation of alcohols using aryl halides as oxidants. An example is shown below:

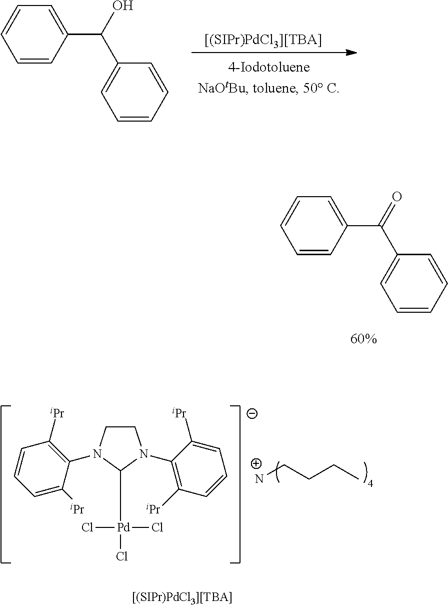

In open-air, sodium tert-butoxide (53 mg, 0.55 mmol), 4-iodotoluene (120 mg, 0.55 mmol), diphenylmethanol (93 mg, 0.5 mmol) and the complex [(SIPr)PdCl₃][TBA] (2.1 mg, 0.5% mol) were added to a 4 ml reaction vial, followed by addition of toluene (1.0 ml). The reaction vial was closed and the reaction mixture was stirred for 2 min at room temperature. Then the vial was set up in a hot stirring plate at 50° C. The reaction progress was monitored at different times by GC and GCMS analysis and by thin layer chromatography. After 24 h, the reaction mixture was concentrated under vacuum, adsorbed in silica and purified using flash chromatography. Eluting from 100% petroleum ether 40/60, further AcOEt gradients (up to 60%) were used. The fractions containing the desired product were combined and concentrated under vacuum leading to a colourless oil identified as benzophenone (100.0. mg, 0.6 mmol, yield 60%).

1H NMR (consistent with literature references): 7.85-7.79 (m, 2H), 7.63-7.57 (m, 1H), 7.52-7.47 (m, 2H).

LCMS (M/Z): Retention time: 18.24 min [M+H]+=183.0.

The mechanism for the above reaction was reported in Berini et al., Org. Lett., 2009, 11, 4244, and is believed to proceed as drawn below:

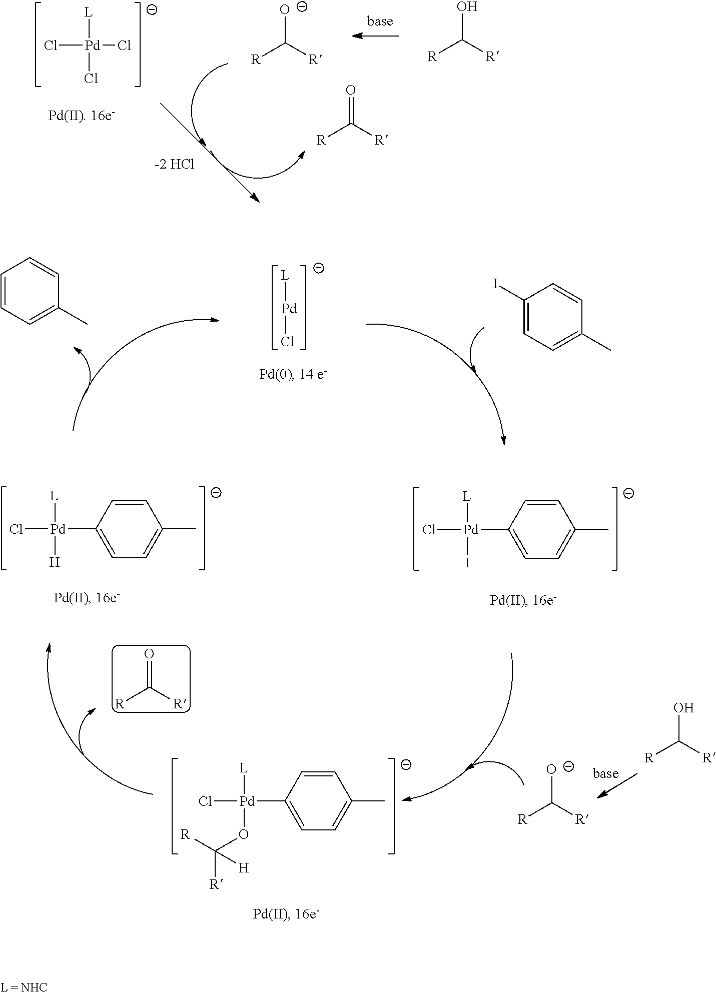

### CATALYTIC EXAMPLE 2

Complexes of the invention may also be used in palladium catalysed C—H activation leading to the formation of a carbon-carbon bond. A proposed reaction scheme is shown below:

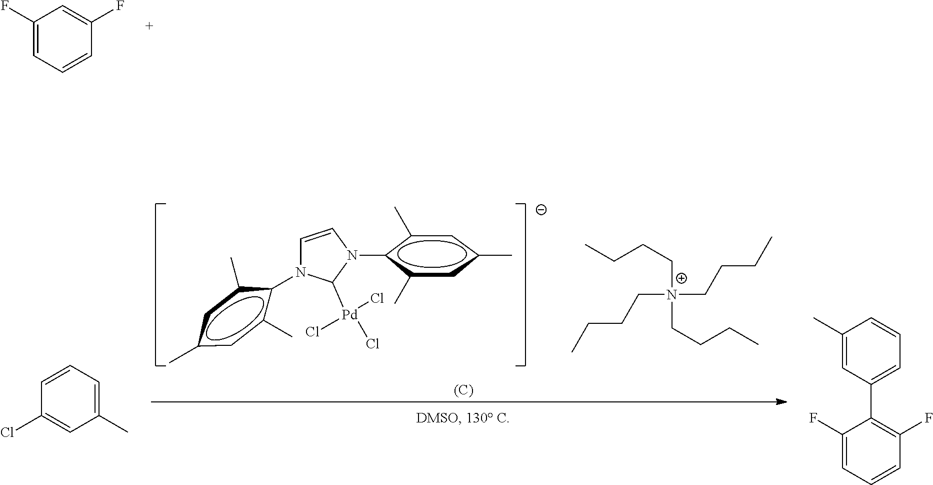

Under an inert atmosphere, 1 mmol of 1,3-difluorobenzene, 1 mmol of m-chlorotoluene, 0.1 mmol of complex C and 2 mL of DMSO will be combined in a reaction vial. The mixture will be heated at 130° C. for 24 h, leading to the formation of 1-phenyl-2,6-difluorobenzene. Progress of the reaction can be monitored by standard techniques, such as thin layer chromatography or LCMS.

C—H activation reactions on various heterocycles has been reported by Vitaku et al., see J. Med. Chem., DOI: 10.1021/jm501100b.

### CATALYTIC EXAMPLE 3

Complexes of the invention may be used in C—H bond activation leading to the formation of a carbon-halide bond. An example is shown below:

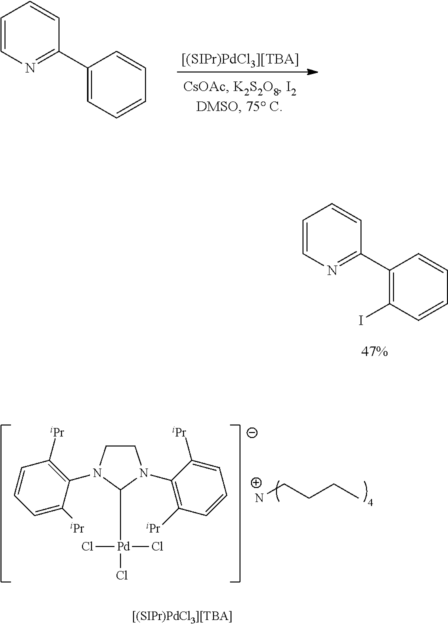

In open air, potassium persulfate (108.1 mg, 0.4 mmol), caesium acetate (92.1 mg, 0.48 mmol), iodine (253.8 mg, 1 mmol), 4 Å molecular sieves (80 mg), the complex [(SIPr)PdCl₃][TBA] (8.5 mg, 5% mol) and DMSO (1.5 ml) were added to a reaction vial. The mixture was stirred at room temperature for 2 min before the addition of 2-phenylpyridine (29 μL, 0.2 mmol), and the reaction vial was closed. The reaction mixture was stirred for 2 min at room temperature and then placed on a hot stirring plate at 75° C. The reaction progress was monitored at different times by GC and GC-MS analysis; the maximum conversion to product after 25 h (GC observed conversion of 62%). At that time, the crude material was washed with brine (40 ml) and extracted with AcOEt. The organic phases were combined, dried using anhydrous Mg₂SO₄, filtered through a sinter and concentrated under vacuum. The crude material was adsorbed in silica and purified using flash chromatography. Eluting from 100% petroleum ether 40/60, further AcOEt gradients (up to 25%) were used. The fractions containing the desired product were combined and concentrated under vacuum leading to a yellow oil corresponding to 2-(2-iodophenyl)pyridine (53.6 mg, 0.19 mmol, yield 47%).

¹H NMR (consistent with literature references): 8.72 (ddd, J=5.0, 1.8, 0.9 Hz, 1H), 8.01-7.95 (m, 1H), 7.78 (td, J=7.7, 1.8 Hz, 1H), 7.53-7.49 (m, 1H), 7.49-7.41 (m, 2H), 7.35-7.29 (m, 1H), 7.12-7.07 (m, J=8.3, 1H).

LCMS (M/Z): Retention time: 12.75 min [M+H]⁺=282.0.

### CATALYTIC EXAMPLE 4

Complexes of the invention may be used in C—H bond activation leading to the formation of a carbon-halide bond. A proposed reaction scheme is shown below:

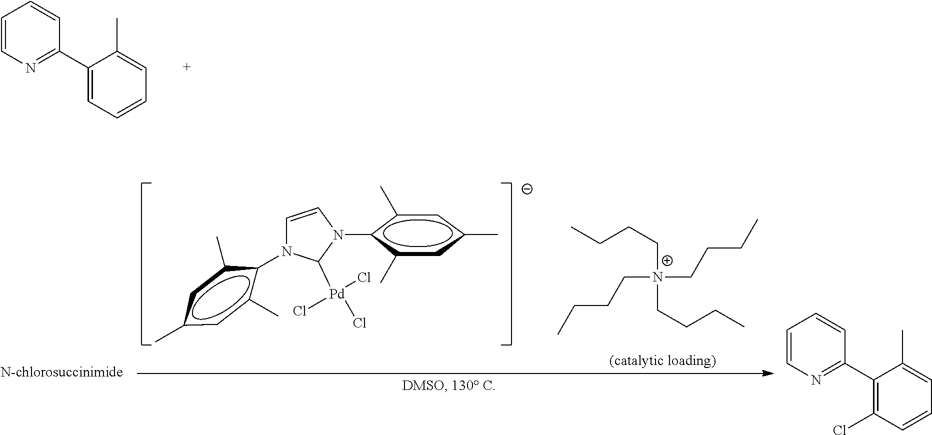

2-(o-Tolyl)pyridine (1 mmol), N-chlorosuccinimide (1 mmol), palladium complex (0.10 mmol) and DMSO (2 mL) will be added to a reaction vial under an inert atmosphere. The mixture will be heated to 130° C. for 24 h, leading to the formation of 2-(2-CI-6-methylphenyl)pyridine. Progress of the reaction can be monitored by standard techniques, such as thin layer chromatography or ¹H NMR.

It is expected that a bromo substituted product could be formed by replacing the N-chlorosuccinamide with N-bromosuccinamide. Similarly, an iodo substituted product should be obtainable by used of N-iodosuccinamide.

### CATALYTIC EXAMPLE 5

Complexes of the invention may be used enantioselective hydrogenation reactions. A proposed reaction scheme is shown below:

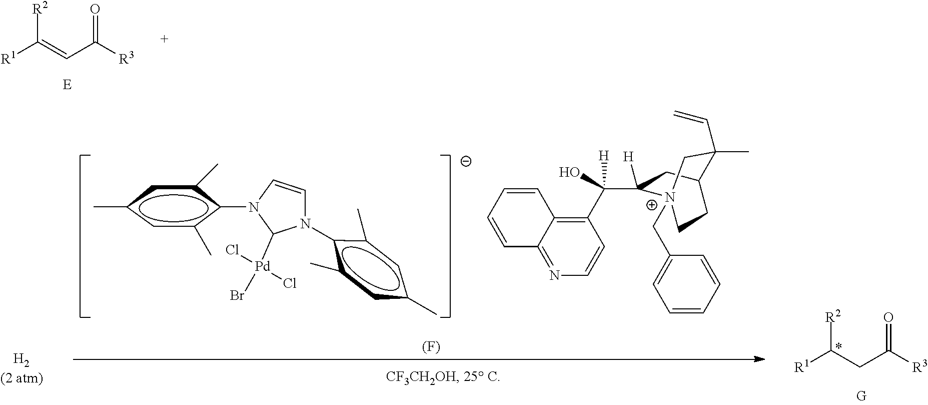

Compound E (1 mmol), complex F (0.1 mmol) and CF₃CH₂OH (2 mL) will be combined in a reaction vessel and subjected to a pressure of H₂ (2 atm), at 25° Cover 24 h, leading to the formation of enantiomerically enriched compound G.

R¹, R² and R³ may be alkyl or aryl groups. Progress of the reaction can be monitored by standard techniques, such as thin layer chromatography or ¹H NMR. The enantiomeric excess of the product can be calculated using known techniques.

### CATALYTIC EXAMPLE 6

A proposed reaction scheme for tandem organic/organometallic catalysis is shown below:

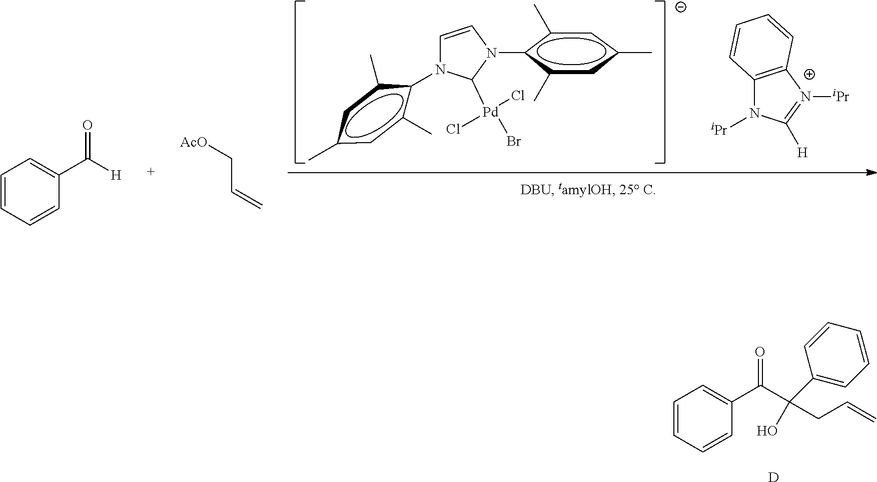

Phenylaldehyde (1 mmol), allyl acetate (1 mmol), palladium complex (0.1 mmol), DBU (1.2 mmol) and tert-amylalcohol (2 mL) will be combined in a reaction vial under an inert atmosphere and the reaction mixture will be stirred at 25° C. for 24 h, leading to the formation of compound D. Progress of the reaction can be monitored by standard techniques, such as thin layer chromatography or ¹H NMR.

### CATALYTIC EXAMPLE 7

Complexes of the invention may be used in C—H bond activation leading to the formation of a carbon-oxygen bond. An example is shown below:

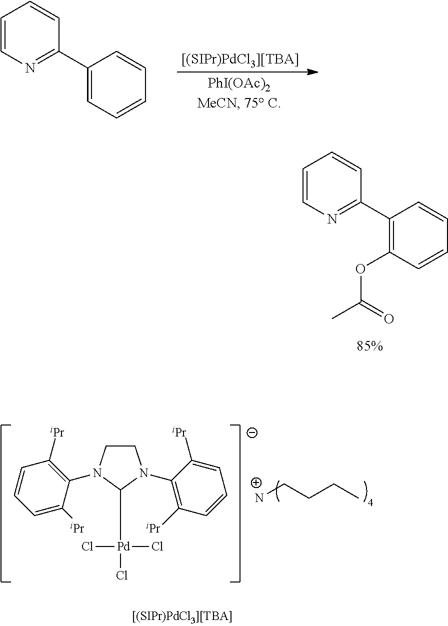

In open air, (diacetoxyiodo)benzene (322.1 mg, 1.0 mmol), the complex [(SIPr)PdCl₃][TBA] (7.6 mg, 1.8% mol) and acetonitrile (1.5 ml) were added to a 4 ml reaction vial. The mixture was stirred at room temperature for 2 min before the addition of 2-phenylpyridine (72 μL, 0.5 mmol). The vial was closed and the reaction mixture was stirred for 2 min at room temperature before placing it in a hot stirring plate at 75° C. The reaction progress was monitored at different times by GC and GC-MS analysis reaching the maximum conversion to product after 48 h (GC observed conversion: 64% starting material to product). At that time, the reaction volume was concentrated under vacuum, adsorbed in silica and purified using flash chromatography. In order to avoid potential hydrolysis of the acetal group as previously reported (see J. Am. Chem. Soc. 2004, 126, 2300-2301), the chromatography column was neutralised using petroleum ether 40/60 (125 ml) and 2 ml of triethylamine. Eluting from 100% petroleum ether 40/60, further AcOEt gradients (up to 85%) were used. The fractions containing the desired product were combined and concentrated under vacuum leading to a colourless oil corresponding to 2-(2-Acetoxylphenyl)pyridine (182.0. mg, 0.85 mmol, yield 85%).

¹H NMR (consistent with literature references): 8.72-8.69 (m, 1H), 7.78-7.68 (m, 2H), 7.57-7.53 (m, 1H), 7.44 (tdd, J=7.5, 1.8, 0.6 Hz, 1H), 7.36 (tdd, J=7.5, 1.2, 0.6 Hz, 1H), 7.28-7.22 (m, 2H), 7.20-7.15 (m, 1H), 2.18 (d, J=0.6 Hz, 3H). LCMS (M/Z): Retention time: 7.97 min [M+H]⁺=214.0.

Without wishing to be bound by theory, it is believed that the catalytic reaction described above takes place via the following catalytic cycle:

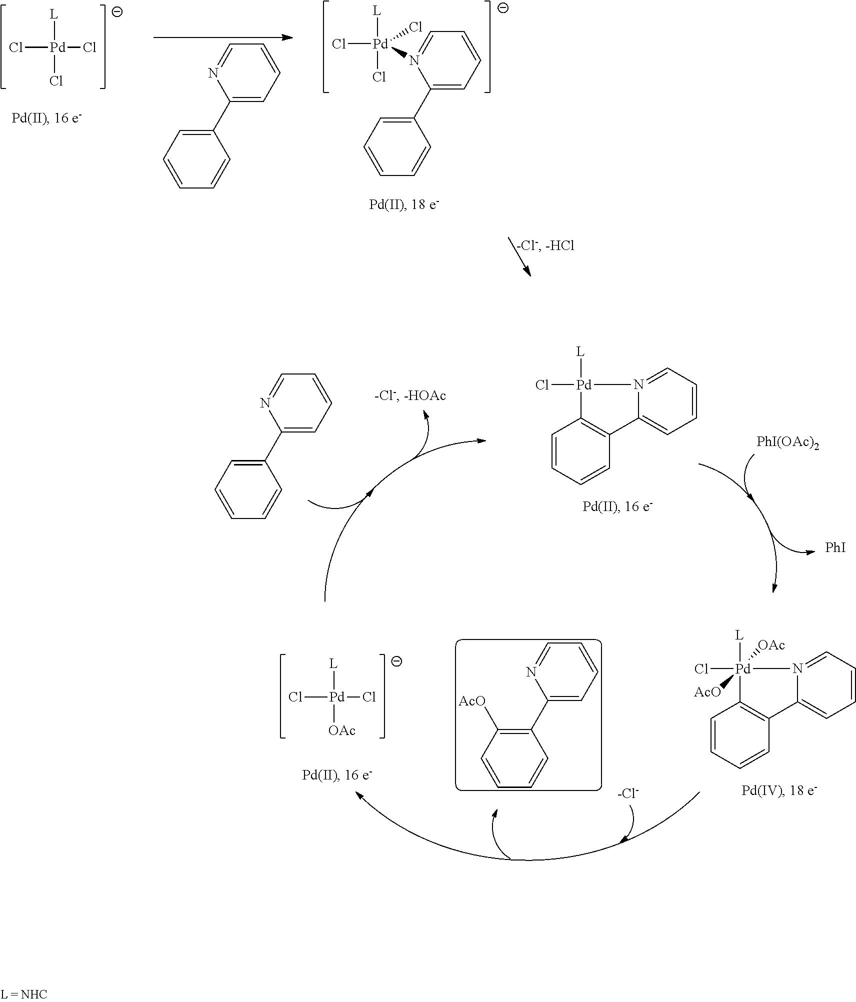

It is interesting to note that this catalytic cycle is thought to utilise the less well characterised Pd(II)/Pd(IV) oxidation states.

### CATALYTIC EXAMPLE 8

Complexes of the invention may be used in Suzuki-Miyaura cross-coupling reactions leading to the formation of a carbon-carbon bond. An example is shown below:

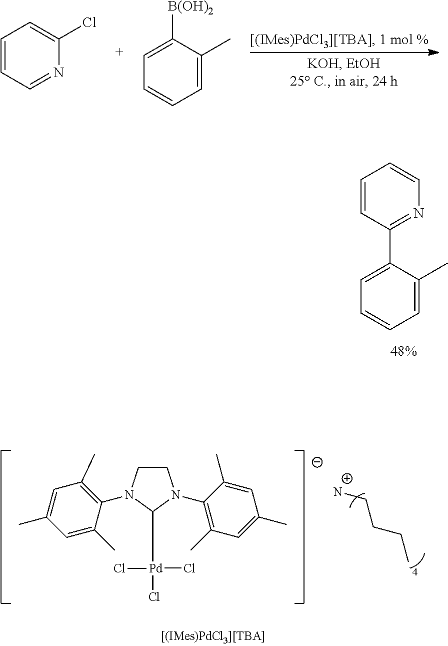

In open air, a 4 mL screw-capped vial equipped with a magnetic stirring bar was charged with potassium hydroxide (0.55 mmol), aryl halide (0.5 mmol), [(IMes)PdCl₃][TBA] (1 mol %), ethanol (1 mL) and the boronic acid (0.55 mmol). The vial was sealed with a screw-cap fitted with a septum and the reaction mixture allowed to stir at room temperature and monitored by gas chromatography. When the reaction was complete or there was no further increase in conversion, it was cooled down to room temperature, poured into water (10 mL) and extracted with Et₂O or EtOAc (3×10 mL). The combined organic layers were washed with water (3×10 mL) and brine (10 mL), dried over MgSO₄ and filtered. The resultant solution was concentrated onto Celite® under reduced pressure for purification by column chromatography. Yield (48%) is an average of two runs.

### CATALYTIC EXAMPLE 9

Complexes of the invention may be used in Buchwald-Hartwig coupling reactions leading to the formation of a carbon-nitrogen bond. An example is shown below:

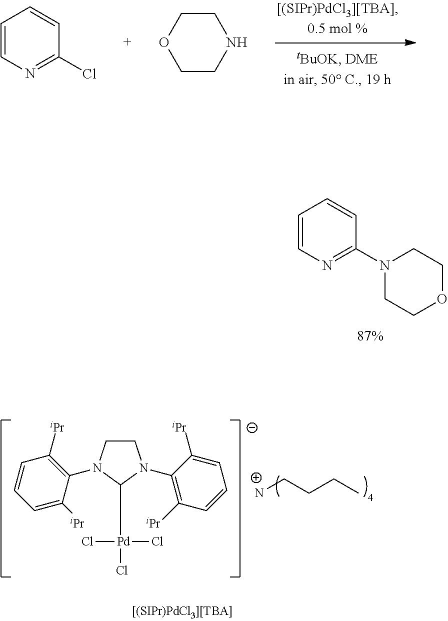

In open air, a 4 mL screw-capped vial equipped with a magnetic stirring bar was charged with potassium tertbutoxide (1.2 mmol), aryl halide (1 mmol), [(SIPr)PdCl₃][TBA] (0.5 mol %), DME (1 mL) and the amine (1.1 mmol). The vial was sealed with a screw-cap fitted with a septum and the reaction mixture allowed to stir at 50° C. for 19 h. Then, it was cooled down to room temperature, poured into water (10 mL) and extracted with Et₂O or EtOAc (3×10 mL). The combined organic layers were washed with water (3×10 mL) and brine (10 mL), dried over MgSO₄ and filtered. The resultant solution was concentrated onto Celite® under reduced pressure for purification by column chromatography. Yield (87%) is an average of two runs.

¹H NMR (500 MHz, Chloroform-d): δ 8.22 (ddd, J=5.2, 2.0, 0.9 Hz, 1H), 7.54 (ddd, J=8.9, 7.2, 2.0 Hz, 1H), 6.74-6.62 (m, 2H), 3.91-3.79 (m, 4H), 3.62-3.47 (m, 4H).

### CATALYTIC EXAMPLE 10

Complexes of the invention may be used in Mizoroki-Heck coupling reactions leading to the formation of a carbon-carbon bond. An example is shown below:

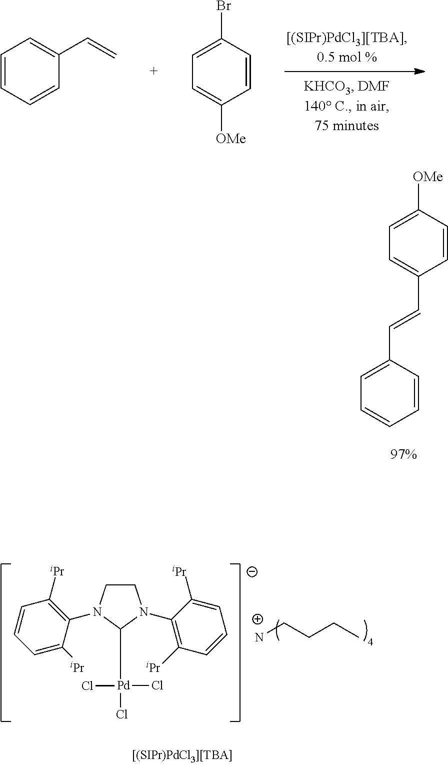

In open air, a 4 mL screw-capped vial equipped with a magnetic stirring bar was charged with potassium bicarbonate (1 mmol), aryl halide (0.5 mmol), [(SIPr)PdCl₃][TBA] (0.5 mol %), anhydrous DMF (1 mL) and the alkene (0.55 mmol). The vial was sealed with a screw-cap fitted with a septum and the reaction mixture allowed to stir at 140° C. and monitored by gas chromatography. When the reaction was complete or there was no further increase in conversion, it was cooled down to room temperature, poured into water (10 mL) and extracted with Et₂O or EtOAc (3×10 mL). The combined organic layers were washed with water (3×10 mL) and brine (10 mL), dried over MgSO₄ and filtered. The resultant solution was concentrated onto Celite® under reduced pressure for purification by column chromatography. Yield (97%) is an average of two runs. Product obtained as a white solid (204 mg, 97%). Gradient eluent: 0:1 to 2:8. EtOAc: 40/60 petroleum ether.

¹H NMR (500 MHz, CDCl₃) δ=7.53-7.45 (m, 3H), 7.35 (t, J=7.7 Hz, 2H), 7.39-7.20 (m, 2H), 7.08 (d, J=16.3 Hz, 1H), 6.99 (d, J=16.3 Hz, 1H), 6.94-6.89 (m, 2H), 3.84 (s, 3H).

1. A complex of formula (I): 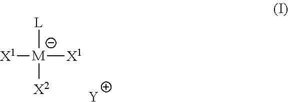
wherein:
M denotes a metal selected from Pd, Pt and Ni;
L denotes an N-heterocyclic carbene ligand;
X¹ denotes F, Cl, Br, I, OH or alkoxide;
X² denotes F, Cl, Br, I, CN, SCN, NCS, N₃ or R^{x}CO₂;
R^{x} denotes H or C₁-C₄ alkyl; and
Y denotes a non-coordinating cation.

2. The complex of claim 1 wherein the N-heterocyclic carbene ligand L is an imidazol-2-ylidene, a tetrahydroimidazol-2-ylidene, or a triazol-5-ylidene.

3. The complex of any preceding claim wherein the complex of formula (I) is a complex of formula (Ia) 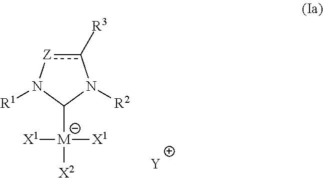
wherein
R¹ and R² independently denote C₁-C₄-alkyl, C₃-C₁₀ cyclohexyl; or C₆-C₁₀ aryl optionally substituted with 1 to 5 R^{a};
Z denotes N, CR⁴ or CHR⁴;
R³ and R⁴ independently denote H, alkyl, aryl, cycloalkyl, alkylaryl, alkyl cycloalkyl, which may be optionally substituted by one or more R^{a}; or
R³ and R⁴ together form a 5-7 membered ring, which may be saturated or unsaturated; or
R² and R³ and/or R¹ and R⁴ may together form an alkylene group having 3 or 4 carbon atoms;
each R^{a} independently denotes halogen, NO₂, C₁-C₄-alkyl, or C₁-C₄-alkoxy; and
M, X¹, X² and Y are as defined in the complex of formula (I).

4. The complex of any preceding claim, wherein M denotes Pd.

5. The complex of any preceding claim, wherein
M denotes Pd;
X¹ denotes F, Cl, Br, I, or C₁-C₄-alkoxide;
X² denotes F, Cl, Br, I, CN, SCN, NCS, N₃ or R^{x}CO₂;
R^{x} denotes H or C₁-C₄ alkyl;
R¹ and R² independently denote C₁-C₄-alkyl, cyclohexyl, adamantyl; or phenyl optionally substituted with 1 to 5 R^{a};
Z denotes CHR⁴ or CR⁴;
R³ and R⁴ independently denote H, C₁-C₄ alkyl, C₁-C₄-alkyl-C₆-C₁₀-aryl, C₁-C₄-alkyl-C₃-C₆-cycloalkyl, which may be optionally substituted by one or more R^{a}; or
R³ and R⁴ may together form —(CH)₄— when Z denotes CR⁴;
each R^{a} independently denotes halogen, NO₂, C₁-C₄-alkyl, or C₁-C₄-alkoxy; and
Y denotes a non-coordinating cation.

6. The complex of any preceding claim, wherein X¹ denotes Cl.

7. The complex of any preceding claim, wherein X² denotes Cl, Br or I.

8. The complex of any preceding claim, wherein Y is chiral.

9. The complex of any preceding claim, wherein Y is a quaternary ammonium compound, a pyridinium cation or an imidazolium cation.

10. The complex of any preceding claim, wherein Y is selected from: 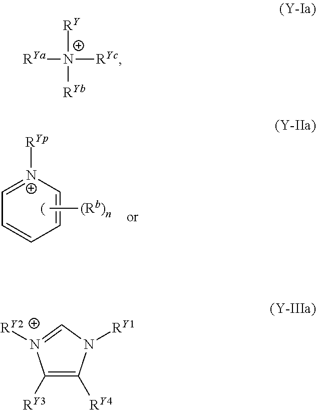
wherein
R^{Y}, R^{Ya}, R^{Yb} and R^{Yc} each independently denote C₁-C₄ alkyl, C₃-C₆ cycloalkyl, C₁-C₄-alkyl-C₃-C₆-cycloalkyl, C₆-C₁₀ aryl or C₁-C₄-alkyl-C₆-C₁₀-aryl, which may be optionally substituted by one or more R^{b}; or
together two of R^{Ya} and R^{Yb} together with the N combine to form a ring containing 4-6 alkylene units; or
R^{Ya}, R^{Yb} and R^{Yc} together with the N combine to form quinuclidine (1-azabicyclo[2.2.2]octane);
R^{Yp} denotes C₁-C₄ alkyl, C₃-C₆ cycloalkyl, C₁-C₄-alkyl-C₃-C₆-cycloalkyl, C₆-C₁₀ aryl or C₁-C₄-alkyl-C₆-C₁₀-aryl, which may be optionally substituted by one or more R^{b};
R^{Y1} and R^{Y2} independently denote C₁-C₄-alkyl, C₃-C₁₀ cycloalkyl; or C₆-C₁₀ aryl optionally substituted with 1 to 5 R^{b};
R^{Y3} and R^{Y4} independently denote H, C₁-C₄ alkyl, C₆-C₁₀ aryl, C₃-C₆ cycloalkyl, C₁-C₄-alkyl-C₆-C₁₀-aryl, or C₁-C₄-alkyl-C₃-C₆-cycloalkyl, which may be optionally substituted by one or more R^{b}; or
R^{Y3} and R^{Y4} together form —(CH)₄—; or
R^{Y2} and R^{Y3} and/or R^{Y1} and R^{Y4} may together form an alkylene group having 3 or 4 carbon atoms;
each R^{b} independently denotes halogen, NO₂, C₁-C₄-alkyl, or C₁-C₄-alkoxy; and
n denotes and integer from 0 to 5.

11. A method for the preparation of a complex of formula (I) comprising reacting a complex of formula (II) with X²Y in a polar solvent: 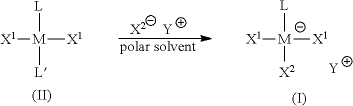
wherein:
M, X¹, X², L and Y are as defined in any preceding claim; and
L′ denotes a tertiary amine.

12. A method according to claim 11, wherein
L′ denotes NR′R″R′″;
wherein
R′, R″ and R′″ each independently denote C₁-C₄ alkyl, C₃-C₆ cycloalkyl or C₆-C₁₀ aryl, which may be optionally substituted by one or more R^{a}; or
together two of R′ and R″ combine to form a ring containing 4-6 alkylene units; or
R′, R″ and R′″ together with the N combine to form quinuclidine (1-azabicyclo[2.2.2]octane) or DABCO (1,4-diazabicyclo[2.2.2]octane); and
each R^{a} independently denotes halogen, NO₂, C₁-C₄-alkyl, or C₁-C₄-alkoxy.

13. The methods of claim 11 or 12 wherein the method is performed at
a temperature of 40° C. or below.

14. Use of a complex according to any of claims 1-10 as a catalyst.

15. Use according to claim 14, wherein the complex of formula (I) or (Ia) is a catalyst for C—H bond activation, preferably leading to the formation of a new carbon-carbon, carbon-halide, carbon-oxygen or carbon-nitrogen bond.

16. Use according to claim 14, wherein the complex of formula (I) or (Ia) is a catalyst for a carbon-carbon bond forming reaction using the M^{II} and M^{IV} oxidation states.

17. Use according to claim 14, wherein the complex of formula (I) or (Ia) is a catalyst for a hydrogenation reaction.

18. Use according to claim 14, wherein the reaction is catalysed by a tandem organic/organometallic catalyst, and wherein the complex of formula (I) or (Ia) forms at least the organometallic catalyst in the tandem catalyst.

19. Use of a complex according to claim 8 as a catalyst for an enantioselective reaction.
